SEQUENCE LISTING (6 sequences)

SEQ ID NO: 1 - DNA, 16 bp, synthetic construct
  misc_feature 1..16: note = oligonucleotide
  modified_base 2: mod_base = m5c
  modified_base 16: mod_base = m5c
  source 1..16: mol_type = other DNA
gcattctaatagcagc

SEQ ID NO: 2 - protein, 30 aa, synthetic construct
  REGION 1..30: note = Synthetic peptide
HAEGTFTSDVSSYLEGQAAKEFIAWLVKGR

SEQ ID NO: 3 - protein, 30 aa, synthetic construct
  REGION 1..30: note = synthetic peptide
  MOD_RES 2: note = Aib
  MOD_RES 6: note = alpha-Me-Phe
  MOD_RES 11: note = alpha-Me-Ser
  MOD_RES 13: note = Lys(Epsilon-(PEG)2-(PEG)2-gammaGlu-Lauroyl)
  MOD_RES 20: note = alpha-Me-Lys
  MOD_RES 22: note = alpha-Me-Phe
  MOD_RES 25: note = Lys(Epsilon-(PEG)2-(PEG)2-gammaGlu-Lauroyl)
HXEGSFTSDVSSKLEGEAAKEFIAKVVEGG

SEQ ID NO: 4 - protein, 30 aa, synthetic construct; identical to SEQ ID NO: 3
  REGION 1..30: note = synthetic peptide
  MOD_RES 2: note = Aib
  MOD_RES 6: note = alpha-Me-Phe
  MOD_RES 11: note = alpha-Me-Ser
  MOD_RES 13: note = Lys(Epsilon-(PEG)2-(PEG)2-gammaGlu-Myristoyl)
  MOD_RES 20: note = alpha-Me-Lys
  MOD_RES 22: note = alpha-Me-Phe
  MOD_RES 25: note = Lys(Epsilon-(PEG)2-(PEG)2-gammaGlu-Myristoyl)

SEQ ID NO: 5 - protein, 30 aa, synthetic construct; identical to SEQ ID NO: 3
  REGION 1..30: note = synthetic peptide
  MOD_RES 2: note = Aib
  MOD_RES 6: note = alpha-Me-Phe
  MOD_RES 11: note = alpha-Me-Ser
  MOD_RES 13: note = Lys(Epsilon-(PEG)2-(PEG)2-gammaGlu-Palmitoyl)
  MOD_RES 20: note = alpha-Me-Lys
  MOD_RES 22: note = alpha-Me-Phe
  MOD_RES 25: note = Lys(Epsilon-(PEG)2-(PEG)2-gammaGlu-Palmitoyl)

SEQ ID NO: 6 - protein, 30 aa, synthetic construct; identical to SEQ ID NO: 3
  REGION 1..30: note = synthetic peptide
  MOD_RES 2: note = Aib
  MOD_RES 6: note = alpha-Me-Phe
  MOD_RES 11: note = alpha-Me-Ser
  MOD_RES 13: note = Lys(Epsilon-(PEG)2-(PEG)2-gammaGlu-Stearoyl)
  MOD_RES 20: note = alpha-Me-Lys
  MOD_RES 22: note = alpha-Me-Phe
  MOD_RES 25: note = Lys(Epsilon-(PEG)2-(PEG)2-gammaGlu-Stearoyl)

(19) United States
(12) Patent Application Publication
(10) Pub. No.: US 2025/0152516 A1
(43) Pub. Date: May 15, 2025

(54) COMPRESSIBLE SODIUM CAPRATE FORMULATIONS

(71) Applicant: AstraZeneca AB, Södertälje (SE)

(72) Inventors: Hanna MATIC, Södertälje (SE); Andreas RÅDEVIK, Södertälje (SE)

(21) Appl. No.: 18/840,222

(22) PCT Filed: Feb. 21, 2023
(86) PCT No.: PCT/IB2023/051570

§ 371 Date: Aug. 21, 2024

Related U.S. Application Data
(60) Provisional application No. 63/312,623, filed on Feb. 22, 2022.

(51) Int. Cl. A61K 9/20; A61K 31/7088; A61K 38/26; C12N 15/113

(52) U.S. Cl. CPC A61K 9/2013; A61K 9/2018; A61K 9/2054; A61K 9/2095; A61K 31/7088; A61K 38/26; C12N 15/1135; C12N 2310/113

(57) ABSTRACT

The present disclosure provides a method of making an oral tablet comprising mixing a macromolecule with sodium caprate to form a mixture, and compressing the mixture into a tablet, wherein the sodium caprate is Form A. The present disclosure also provides a tablet made by the process provided herein. Also provided is a composition comprising sodium caprate and a macromolecule.

### FIELD OF THE DISCLOSURE

The present disclosure provides a method of making an oral tablet comprising mixing a macromolecule with particular forms of sodium caprate to form a mixture and compressing the mixture into a tablet. Also provided are methods of making an oral tablet comprising mixing a macromolecule with Form A sodium caprate to form a mixture, and compression the mixture into a tablet. The present disclosure also provides a tablet made by the processes provided herein. Also provided are compositions comprising sodium caprate and a macromolecule. The present disclosure also relates to compositions comprising Form A sodium caprate and a macromolecule.

### BACKGROUND

Tablets have been a popular pharmaceutical dosage form due to their convenience for the patient, accurate dosage, compactness and portability, blandness of taste, ease of administration, and elegant, distinctive appearance. Tablets may be plain, film or sugar coated, layered, bisected, embossed, and/or sustained release. Tablets may be made in a variety of sizes, shapes, and colors. Oral tablets (i.e., tablets intended for oral administration) may be swallowed, chewed, or dissolved in the buccal cavity or beneath the tongue.

In general, a tablet contains an active or therapeutic ingredient, and additionally typically comprises any number of inert materials known as excipients. The excipients may be classified according to their role in the final tablet. Examples of excipients include permeability enhancers, binders, lubricants, glidants, coloring agents, flavoring agents, diluents, disintegrants, and the like. Examples of pharmaceutical excipients can be found, e.g., in the American Pharmaceutical Review Pharmaceutical Excipients database.

For production, a tablet tensile strength of at least 1.7 MPa is typically desired when preparing a tablet (see, e.g., Pitt et al., Powder Tech 238:169-175 (2013)). Tablets with tensile strength of 1.0 to 1.7 MPa are acceptable but may require additional care in handling. Thus, tablet excipients and their concentrations are taken into consideration to achieve favorable tensile strength. When a particular ingredient is present at a high concentration in a tablet, the ingredient is evaluated for its mechanical and tableting properties along with sticking propensity, which may be dependent upon its crystal structure and size and/or shape of its particles. For example, sodium caprate is sometimes used in oral tablets as a permeability enhancer, in some cases at high concentrations. In such cases, the tableting properties of sodium caprate need to be acceptable.

### SUMMARY OF THE DISCLOSURE

The inventors found that tablets comprising compositions comprising particular forms of sodium caprate have unexpected benefits of higher physical integrity, e.g., higher hardness, and less “stickiness” to the tableting machinery, e.g., tableting dies and presses. For example, in some embodiments, the present disclosure is directed to compositions comprising a macromolecule and sodium caprate, wherein the sodium caprate is Form A sodium caprate characterized by a X-Ray Powder Diffraction (XRPD) spectrum which includes a peak at 4° 2θ and/or by wide-angle X-ray scattering (WAXS) spectrum which includes a peak at a region of 0.1 to 0.15 Å⁻¹, and with peaks at 0.12 and 0.23 Å⁻¹. In some embodiments, the sodium caprate is Form A sodium caprate.

Thus, the present disclosure is directed to a method of making an oral tablet comprising particular forms of sodium caprate. In some embodiments, the disclosure provides a method of making an oral tablet, the method comprising: (a) mixing a macromolecule with Form A sodium caprate to form a mixture; and (b) direct compressing the mixture to form a tablet.

In some embodiments, the sodium caprate is Form A sodium caprate characterized by a small-angle X-ray scattering (SAXS) spectrum which includes a peak at a region of 0.1 to 0.15 Å⁻¹. In some embodiments, Form A sodium caprate is characterized by a SAXS peak at 0.12 and 0.23 Å⁻¹.

In some embodiments, the sodium caprate is Form A sodium caprate characterized by an X-ray powder diffraction (XRPD) spectrum which includes a peak at 4° 2θ.

In some embodiments, the sodium caprate is Form A sodium caprate characterized by a wide-angle X-ray scattering (WAXS) spectrum which includes a peak at a region of 0.1 to 0.15 Å⁻¹. In some embodiments, the sodium caprate is Form A sodium caprate characterized by a wide-angle X-ray scattering (WAXS) spectrum which includes a peak at 0.12 and 0.23 Å⁻¹.

In some embodiments, the sodium caprate has a water content of less than about 3.5% as measured by Karl Fischer titration. In some embodiments, the sodium caprate has a water content of less than about 2% as determined by Karl Fischer titration. In some embodiments, the sodium caprate has a water content of less than about 1.5% as determined by Karl Fischer titration.

In some embodiments, the sodium caprate of step (a) is sieved or milled to obtain a desired particle size before the direct compression of (b) to form a tablet. In some embodiments, the mixture of step (a) is sieved or milled to obtain a desired particle size before the direct compression of (b) to form a tablet. In some embodiments, the sodium caprate has a particle size of about 10 μm to about 1300 μm before step (b). In some embodiments, the sodium caprate has a particle size of about 35 μm to about 850 μm before step (b). In some embodiments, the sodium caprate has a particle size of about 50 μm to about 700 μm before step (b).

In some embodiments, after the tablet compression of (b), a coating is applied on the tablet.

In some embodiments, the method does not comprise a milling step after the mixing of step (a). In some embodiments, the direct compressing is performed a range of about 15 MPa to 500 MPa. In some embodiments, the direct compressing is performed at 25 MPa to 300 MPa. In some embodiments, the direct compressing is performed at 50 MPa to 200 MPa.

In some embodiments, the mixing of (a) further comprises a polyol. In some embodiments, the polyol is sorbitol, mannitol, maltitol, xylitol or combinations thereof. In some embodiments, the polyol is mannitol. In some embodiments, the mixing of (a) further comprises additional excipients, e.g. glidants and/or lubricants and/or other fillers and/or disintegrants.

Non-limiting examples of pharmaceutically acceptable excipients include microcrystalline cellulose, Dicalcium phosphate, Lactose, Mannitol, Sodium stearyl fumarate (PRUV), Magnesium stearate, Silica colloidal hydrated, Crospovidone, Sodium croscarmellose, Sodium bicarbonate, low-substituted hydroxypropylcellulose (L-HPC), Sodium starch glycolate, Water, Ethanol, Isopropyl alcohol or other solvents, Polyvinylpyrrolidone (PVP), Hydroxy propyl cellulose (HPC), hydroxypropylmethylcellulose (HPMC), (tromethamine) (TRIS), any salt of carbonate, borate, phosphate, Tartaric acid, Magnesium hydroxide, Magnesium oxide, Sodium bicarbonate, Propyl gallate, alpha-tocopherol, butylated hydroxy anisole (BHA), ascorbic acid, Solutol, polysorbate 80, and ethylenediaminetetraacetic acid (EDTA).

In some embodiments, the macromolecule is about 0.1% to about 12% by weight of the tablet. In some embodiments, the sodium caprate is about 30% to about 99% by weight of the tablet. In some embodiments, the additional excipients range from about 0.001% to about 50% by weight of the tablet.

In some embodiments, the macromolecule is about 0.1% to about 12% by weight of the tablet. In some embodiments, the sodium caprate is about 30% to about 99% by weight of the tablet. In some embodiments, the polyol is about 0.001% to about 50% by weight of the tablet. In some embodiments, the additional excipients range from about 0.001% to about 50% by weight of the tablet.

In some embodiments, the macromolecule is a peptide or an oligonucleotide. In some embodiments, the macromolecule is an oligonucleotide. In certain embodiments, the oligonucleotide is 8 to 100 nucleotides in length, for example 10 to 80 nucleotides in length, such as 12 to 40 nucleotides in length, for instance, 15 to 30 nucleotides in length, such as about 12 nucleotides, about 15 nucleotides, about 18 nucleotides, or about 20 nucleotides in length. In certain embodiments, the oligonucleotide is modified, for example, the oligonucleotide comprises at least one modified internucleoside linkage, at least one modified sugar, or at least one modified nucleobase. In certain embodiments, the oligonucleotide is single-stranded or double-stranded. In certain embodiments, the oligonucleotide is an antisense nucleotide, miRNA antagonist or miRNA mimic.

In embodiments, the oligonucleotide is an antisense oligonucleotide targeting metastasis-associated lung adenocarcinoma transcript 1 (MALAT1) comprising at least one nucleic acid with a locked sugar modified moiety (“LNA”). In such embodiments, the antisense oligonucleotide targets MALAT1 and has the sequence: GM5CAttctaatagcAGM5C, where m5c is 5-methylcytidine and capital letters are LNA nucleosides (SEQ ID NO:1). In embodiments, the oligonucleotide is an antisense oligonucleotide that targets MALAT1 and reduces expression of MALAT1 in cells by about 1% to about 100%. In embodiments, the oligonucleotide is an antisense oligonucleotide that targets MALAT1 and reduces expression of MALAT1 in cells by about 1%, by about 10%, by about 20%, by about 30%, by about 40%, by about 50%, by about 60%, by about 70%, by about 80%, by about 90%, by about 95% or by about 100%.

In certain embodiments, the macromolecule is a peptide. In certain embodiments, the peptide is 5 to 3000 amino acids in length or 10 to 2000 amino acids in length, such as 10 to 150 amino acids in length, for example, 20 to 100 amino acids in length, for instance, 30 to 75 amino acids in length, such as about 20 amino acids, about 30 amino acids, about 40 amino acids, about 50 amino acids, about 60 amino acids, about 70 amino acids, about 80 amino acids, about 90 amino acids, or about 100 amino acids in length.

In certain embodiments, the peptide comprises the amino acid sequence HAibEGS-(αMeF)TSDV-(αMeS)SX13LE-GEAA(αMeK)-E(αMeF)IAX25-VVEGG
wherein: X13 is a lipid modified K; X25 is a lipid modified K; and where “αM” is an alpha-methyl functionalized amino acid, and wherein the two lipid modified K residues are the same or are different, and are selected from the group consisting of: K(ε-(PEG)₂-(PEG)₂-γE-Lauroyl), K(ε-(PEG)₂-(PEG)₂-γE-Myristoyl), K(ε-(PEG)₂-(PEG)₂-γE-Palmitoyl), K(ε-(PEG)₂-(PEG)₂-γE-Stearoyl), and any combination thereof.

In some embodiments, the oral tablet has a tablet tensile strength of about 0.5 to 5.0 MPa. In some embodiments, the oral tablet has a tablet tensile strength of about 0.75 MPa to about 3 MPa. In at least one embodiment, the tablet tensile strength from about 1 MPa to about 2.5 MPa.

In some embodiments, the disclosure is directed to a method of making an oral tablet, the method comprising: (a) sieving or milling sodium caprate to obtain sodium caprate particles having a particle size ranging from about 10 μm to about 1300 μm, such as ranging from about 35 μm to about 850 μm, for instance, ranging from about 50 μm to about 750 μm, (b) mixing the sodium caprate particles with a macromolecule and other excipients to form a mixture, and (c) direct compressing the mixture of to form a tablet, wherein the sodium caprate is Form A sodium caprate characterized by a X-ray powder diffraction (XRPD) spectrum which includes a peak at 4° 2θ and/or by a wide-angle X-ray scattering (WAXS) spectrum which includes a peak at a region of 0.1 to 0.15 Å⁻¹ and with peaks at 0.12 and 0.23 Å⁻¹ and wherein the method does not comprise a wet granulation step, a drying step or a milling step after the mixing of step (b).

In some embodiments, the disclosure is directed to a tablet made by the methods described herein.

In some embodiments, the disclosure is directed to a method of making an oral tablet, the method comprising: a) mixing sodium caprate particles with additional excipients to form a mixture; b) granulating the mixture with an organic non-aqueous solvent to form granules; c) drying the granules; d) sieving or milling the granules to obtain granules having a particle size of about 100 μm to 2000 μm; e) blending with a macromolecule and/or additional excipients; f) compressing the mixture to form a tablet, wherein the sodium caprate after step c) is Form A sodium caprate. In some embodiments, Form A sodium caprate is characterized by an X-ray powder diffraction (XRPD) spectrum which includes a peak at 4° 2θ. In some embodiments, the organic solvent is chosen from methanol (MeOH), ethanol (EtOH), 2-propanol (IPA), toluene, cyclohexane, dioxane, dimethylformamide (DMF), acetonitrile (ACN), tetrahydrofuran (THF), methyl isobutyl ketone (MIBK), ethyl acetate (EtOAc), and diethylether. In at least one embodiment, the organic solvent is ethanol. In some embodiments, the additional excipient is a polyol. In some embodiments, the polyol is mannitol.

In some embodiments, the disclosure is directed to a method of making an oral tablet, the method comprising: a) granulating sodium caprate particles with an organic non-aqueous solvent to form granules; b) drying the granules; c) sieving or milling the granules to obtain granules having a particle size of about 100 μm to 2000 μm; and d) blending the granules with a macromolecule and/or additional excipients to form a mixture; e) compressing the mixture to form a tablet wherein the sodium caprate after step (b) is Form A sodium caprate. In some embodiments, Form A sodium caprate is characterized by a X-ray powder diffraction (XRPD) spectrum which includes a peak at 4° 2θ and/or by a wide-angle X-ray scattering (WAXS) spectrum which includes a peak at a region of 0.1 to 0.15 Å⁻¹ and with peaks at 0.12 and 0.23 Å⁻¹. In some embodiments, the organic solvent is chosen from methanol (MeOH), ethanol (EtOH), 2-propanol (IPA) toluene, cyclohexane, dioxane, dimethylformamide (DMF), acetonitrile (ACN), tetrahydrofuran (THF), methyl isobutyl ketone (MIBK), ethyl acetate (EtOAc), and diethylether. In at least one embodiment, the organic solvent is ethanol. In some embodiments, the additional excipient is a polyol. In some embodiments, the polyol is mannitol.

In some embodiments, the disclosure is directed to a method of making an oral tablet, the method comprising: a) mixing sodium caprate particles with additional excipients and a macromolecule to form a mixture; b) granulating the mixture with an organic non-aqueous solvent to form granules; c) drying the granules; d) sieving or milling the granules to obtain granules having a particle size of about 100 μm to 2000 μm; e) blending the granules with additional excipients to form a mixture; f) compressing the mixture to form a tablet, wherein the sodium caprate after step c) is Form A sodium caprate. In some embodiments, sodium caprate is characterized by an X-ray powder diffraction (XRPD) spectrum which includes a peak at 4° 2θ. In some embodiments, the organic non-aqueous solvent is chosen from methanol (MeOH), ethanol (EtOH), 2-propanol (IPA), toluene, cyclohexane, dioxane, dimethylformamide (DMF), acetonitrile (ACN), tetrahydrofuran (THF), methyl isobutyl ketone (MIBK), ethyl acetate (EtOAc), and diethylether. In at least one embodiment, the organic non-aqueous solvent is ethanol. In at least one embodiment, the organic non-aqueous solvent is isopropyl alcohol.

In some embodiments, the disclosure is directed to a composition comprising a macromolecule and sodium caprate, wherein the sodium caprate is Form A sodium caprate characterized by a X-ray powder diffraction (XRPD) spectrum which includes a peak at 4° 2θ and/or by a wide-angle X-ray scattering (WAXS) spectrum which includes a peak at a region of 0.1 to 0.15 Å⁻¹ and with peaks at 0.12 and 0.23 Å⁻¹. In some embodiments, the composition further comprises a polyol. In some embodiments, the polyol is mannitol. In some embodiments, the macromolecule in the composition is about 0.1% to about 12% by weight of the tablet. In some embodiments, the sodium caprate in the composition is about 30% to about 99% by weight of the tablet. In some embodiments, the polyol in the composition is about 0.001% to about 50% by weight of the tablet. In some embodiments, the macromolecule in the composition is a peptide or an oligonucleotide. In some embodiments, the macromolecule in the composition is an oligonucleotide. In some embodiments, the macromolecule in the composition is a compound comprising a modified oligonucleotide. In some embodiments, the macromolecule in the composition is a compound comprising the sequence of SEQ ID NO: 1. In some embodiments, the macromolecule in the composition is a compound comprising a modified peptide. In some embodiments, the macromolecule in the composition is a compound comprising a modified peptide of SEQ ID NOS: 3-6.

In some embodiments, the composition is in the form of an oral tablet, and the oral tablet has a tablet tensile strength ranging from about 0.5 MPa to about 5.0 MPa, such as ranging from about 0.75 MPa to about 3 MPa. In at least one embodiment, the tablet tensile strength from about 1 MPa to about 2.5 MPa

### BRIEF DESCRIPTION OF THE DRAWINGS

FIG. 1 relates to Example 1. FIG. 1 shows X-ray powder diffraction (XRPD) patterns for sodium caprate obtained from five different suppliers.

FIGS. 2A-2D relate to Example 1. FIG. 2A shows the XRPD pattern for Form A sodium caprate, FIG. 2B shows the XRPD pattern for Form B sodium caprate, FIG. 2C shows the XRPD pattern for Form C sodium caprate, and FIG. 2D shows the XRPD pattern for Form D sodium caprate.

FIG. 3A-3D relate to Example 2. FIG. 3A shows the wide-angle X-ray scattering (WAXS) spectrum for Form A sodium caprate, FIG. 3B shows the wide-angle X-ray scattering (WAXS) spectrum for Form B sodium caprate, FIG. 3C shows the wide-angle X-ray scattering (WAXS) spectrum for Form C sodium caprate and FIG. 3D shows the wide-angle X-ray scattering (WAXS) spectrum for Form D sodium caprate.

FIG. 4A to 4D relates to Example 3. FIG. 4A shows the small-angle X-ray scattering (SAXS) spectra for Form A sodium caprate, FIG. 4B shows the small-angle X-ray scattering (SAXS) spectra for Form B sodium caprate, FIG. 4C shows the small-angle X-ray scattering (SAXS) spectra for Form C sodium caprate, and FIG. 4D shows the small-angle X-ray scattering (SAXS) spectra for Form D.

FIGS. 5A-5C relate to Example 5. FIGS. 5A-5C show compaction properties of Forms A, B, C, and D sodium caprate as measured by tablet tensile strength. FIG. 5A shows particle sizes of about 37 to about 53 μm, FIG. 5B shows particle sizes of about 150 to about 300 μm, and FIG. 5C shows particle sizes of about 600 to about 710 μm.

FIG. 6 relates to Example 6. FIG. 6 shows XRPD patterns of sodium caprate treated with different solvents. The solvent lists from bottom to top: diethylether (Form A), EtOAc (Form A), MIBK (Form A), THF (Form A), acetone (Form A), can (Form A), DMF (Form A), DMSO (amorphous), dioxane (Form A) cyclohexane, toluene, EtOH (Form A), MeOH (Form A), water (Form C). Abbreviations used-MeOH: methanol; EtOH: ethanol; DMSO: dimethylsulfoxide; DMF: dimethylformamide; ACN: acetonitrile; THF: tetrahydrofuran; MIBK: methyl isobutyl ketone; EtOAc: ethyl acetate.

FIG. 7 relates to Example 6. FIG. 7 shows XRPD patterns of sodium caprate treated with different solvents. The solvent lists from bottom to top: acetone (Form A), acetonitrile (Form A), EtOH (Form A), toluene (Form A), water (Form C).

FIG. 8 relates to Example 7. FIG. 8 shows compaction properties of sodium caprate wet granulated with ethanol and granulated with water as measured by tablet tensile strength.

FIG. 9 relates to Example 7. FIG. 9 shows compaction properties of Form A sodium caprate, Form B sodium caprate, Form C sodium caprate, and Form D sodium caprate after wet granulation with ethanol; and Form A sodium caprate, Form C sodium caprate, and Form D sodium caprate after wet granulation with 2-propanol as measured by tablet tensile strength.

FIGS. 10A-10G relate to Example 7. FIG. 10A shows the WAXS spectrum for Form A sodium caprate granulated with ethanol, FIG. 10B shows the WAXS spectrum for Form B sodium caprate granulated with ethanol, FIG. 10C shows the WAXS spectrum for Form C sodium caprate granulated with ethanol, FIG. 10D shows the WAXS spectrum for Form D sodium caprate granulated with ethanol, FIG. 10E shows the WAXS spectrum for Form A sodium caprate granulated with 2-propanol, FIG. 10F shows the WAXS spectrum for Form C sodium caprate granulated with 2-propanol, and FIG. 10G shows the WAXS spectrum for Form D sodium caprate granulated with 2-propanol.

FIGS. 11A-11G relate to Example 7. FIG. 11A shows the XRPD pattern for Form A sodium caprate granulated with ethanol, FIG. 11B shows the XRPD pattern for Form B sodium caprate granulated with ethanol, FIG. 11C shows the XRPD pattern for Form C sodium caprate granulated with ethanol, FIG. 11D shows the XRPD pattern for Form D sodium caprate granulated with ethanol, FIG. 11E shows the XRPD pattern for Form A sodium caprate granulated with 2-propanol, FIG. 11F shows the XRPD pattern for Form C sodium caprate granulated with 2-propanol, and FIG. 11G shows the XRPD pattern for Form D sodium caprate granulated with 2-propanol.

FIG. 12 relates to Example 8 and illustrates an exemplary process for directed compression tablet production.

FIG. 13 relates to Example 8. FIG. 13 shows the tabletability of sodium caprate Form A wet granulated with ethanol dried and milled and mixed with either microcrystalline cellulose (MCC) or hydroxy propyl cellulose (L-HPC) and with and without sodium stearyl fumarate (PRUV).

FIG. 14 relates to Example 9, Example 10 and Example 13 (Formulation 2) and illustrates an exemplary process for tablet production that include wet granulation of sodium caprate and other excipients, and in which the macromolecule, e.g., MALAT1 ASO or MEDI7219 Peptide, is not subject to wet granulation and drying.

FIG. 15 relates to Example 10. FIG. 15 shows compaction properties of Form A sodium caprate and mannitol, Form B sodium caprate and mannitol, Form C sodium caprate and mannitol, and Form D sodium caprate and mannitol after wet granulation with ethanol; and Form A sodium caprate and mannitol and Form D sodium caprate and mannitol after wet granulation with 2-propanol, as measured by tablet tensile strength.

FIGS. 16A-16F relate to Example 10. FIG. 16A shows the WAXS spectrum for Form A sodium caprate and mannitol granulated with ethanol, FIG. 16B shows the WAXS spectrum for Form B sodium caprate and mannitol granulated with ethanol, FIG. 16C shows the WAXS spectrum for Form C sodium caprate and mannitol granulated with ethanol, FIG. 16D shows the WAXS spectrum for Form D sodium caprate and mannitol granulated with ethanol, FIG. 16E shows the WAXS spectrum for Form A sodium caprate and mannitol granulated with 2-propanol, and FIG. 16F shows the WAXS spectrum for Form D sodium caprate and mannitol granulated with 2-propanol.

FIGS. 17A-17F relate to Example 10. FIG. 17A shows the XRPD pattern for Form A sodium caprate and mannitol granulated with ethanol, FIG. 17B shows the XRPD pattern for Form B sodium caprate and mannitol granulated with ethanol, FIG. 17C shows the XRPD pattern for Form C sodium caprate and mannitol granulated with ethanol, FIG. 17D shows the XRPD pattern for Form D sodium caprate and mannitol granulated with ethanol,

FIG. 17E shows the XRPD pattern for Form A sodium caprate and mannitol granulated with 2-propanol, and FIG. 17F shows the XRPD pattern for Form D sodium caprate and mannitol granulated with 2-propanol.

FIG. 18 relates to Example 12 (Formulation 1). FIG. 18 illustrates an exemplary process for tablet production that include wet granulation for sodium caprate, and in which neither the macromolecule nor the other excipients are subject to wet granulation and drying.

FIG. 19 illustrates an exemplary process for tablet production that include wet granulation of sodium caprate together with an exemplary macromolecule and other excipients.

FIGS. 20A-20B relate to Example 14. FIG. 20A shows the dissolution profile of tablets comprising Form A sodium caprate and MALAT1 ASO. FIG. 20B shows the dissolution profile of tablets comprising Form A sodium caprate and MEDI7219 Peptide.

FIG. 21 relates to Example 15. FIG. 21 illustrates an exemplary process for preparation of spherical agglomerates of sodium caprate.

### DETAILED DESCRIPTION OF THE DISCLOSURE

The present disclosure relates to methods of making oral tablets.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the present disclosure. Furthermore, compositions, systems, host cells, and/or vectors of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, the term “macromolecule” refers to a natural and/or synthetic molecule, typically polymeric and comprises thousands or more atoms. Macromolecules can include, e.g., biopolymers such as nucleic acids, proteins, carbohydrates, and polymeric lipids, and large, non-polymeric molecules such as, e.g., lipids, macrocycles, and large-molecule drugs, e.g., a drug molecule conjugated to a macromolecule such as a peptide or antibody. In some embodiments, the macromolecules of the present disclosure are therapeutic macromolecules. In some embodiments, the therapeutic macromolecule is a therapeutic peptide, a protein, or an antibody or conjugate or derivative thereof; a therapeutic oligonucleotide such as an antisense oligonucleotide; or a water-soluble polymer or polymer-drug conjugate such as the ones described in Yang et al., J Control Release 0:288-303 (2014).

As used herein, the terms “nucleic acid,” “nucleic acid molecule,” “oligonucleotide,” or “polynucleotide” refer to a polymeric compound comprising covalently linked nucleotides, each of which may be independently modified or unmodified. Oligonucleotides can comprise naturally-occurring nucleobases, e.g., adenine (A), guanine (G), cytosine (C), uracil (U), or thymine (T); a modified nucleobase, e.g., hypoxanthine, xanthine, or 5-methylcytosine; or synthetic nucleobases, e.g., d5SICS, dNaM, aminoallyl, isoguanine, isocytosine, or the Hachimoji nucleobases described in Hoshika et al., Science 363:884-887 (2019). Oligonucleotides can include RNA, DNA, XNA, and/or RNA-DNA hybrids, all of which may be single-stranded or double-stranded. In some embodiments, an oligonucleotide of the present disclosure is a therapeutic oligonucleotide. In some embodiments, an oligonucleotide of the present disclosure is an antisense oligonucleotide. In some embodiments, the antisense oligonucleotide is antisense RNA.

The terms “peptide,” “peptide,” and “protein” are used interchangeably herein, and refer to a polymeric form of amino acids of any length, which can include coded and non-coded amino acids, chemically or biochemically modified or derivatized amino acids, and peptides having modified peptide backbones. In some embodiments, a peptide or peptide of the present disclosure is a therapeutic peptide such as the ones described in Marqus et al., J Biomed Sci 24:21 (2017). In some embodiments, a peptide or peptide of the present disclosure is a therapeutic protein. Therapeutic proteins include, e.g., antibodies or conjugates or derivatives thereof; Fc fusion proteins; anticoagulants, blood factors, bone morphogenetic factors; engineered protein scaffolds; enzymes; growth factors; hormones; interferons; interleukins; and thrombolytics. See, e.g., Dimitrov, Methods Mol Biol 899:1-26 (2012) for further examples of therapeutic proteins.

The term “alpha-methyl functionalized amino acids” refer to amino acids in which the first (alpha) carbon atom of the amino acid includes a methyl group (CH₃) substituent bound to the alpha carbon. Alpha-methyl functionalized amino acids include any of the naturally occurring twenty amino acids that include such a functionalization. Alpha-methyl functionalized amino acids can replace any native amino acid in a peptide. The term “native” amino acid refers to one of the standard 20 amino acids that exist in biologically generated proteins

The term “synthetic peptide” or “synthetic peptide” refer to a polymer of amino acid residues that has been generated by chemically coupling a carboxyl group or C-terminus of one amino acid to an amino group or N-terminus of another. Chemical peptide synthesis typically starts at the C-terminus of the peptide and ends at the N-terminus. Various methods for generating synthetic peptides are well known in the art.

The term “polyol” refers to a compound containing multiple hydroxyl groups and, in some cases, may also be referred to as a “sugar alcohol.” Non-limiting examples of polyols include mannitol, maltitol, sorbitol, xylitol, erythritol, isomalt, and the like. In some embodiments, a polyol is a pharmaceutical excipient. In some embodiments, a polyol is included in an oral tablet described herein.

The term “particle size” as used herein refers to the median or the average dimension of particles in a sample and may be based on the number of particles, the volume of particles, or the mass of particles, and may be obtained using any number of standard measurement techniques known in the art. A desired particle size range material may be obtained directly from a synthesis process, or any known particle size reduction processes can be used, such as but not limited to: sifting, milling, micronization, fluid energy milling, media milling, ball milling, milled through high pressure homogenizer, air jet milling, and the like. Methods for determining particle size include analytical sieving and laser light diffraction, such as using equipment from Malvern Instruments Ltd. (Malvern, Worcestershire, United Kingdom).

As described herein, a macromolecule is a natural and/or synthetic molecule, typically polymeric and comprises thousands or more atoms. In some embodiments, the macromolecule is a nucleic acid, a protein, a carbohydrate, or a lipid. In some embodiments, the macromolecule is a peptide. In some embodiments, the macromolecule is a therapeutic peptide, a protein, or an antibody or conjugate or derivative thereof.

In some embodiments, the macromolecule is an oligonucleotide. In some embodiments, the macromolecule is an RNA molecule. In some embodiments, the macromolecule is an antisense oligonucleotide. In some embodiments, the oligonucleotide can be 5 to 1000 nucleotides in length, 10 to 500 nucleotides in length, or 10 to 100 nucleotides in length. Various therapeutic oligonucleotides are known in the art and can be suitable to be used with the disclosure herein.

In some embodiments, the oligonucleotide is an antisense oligonucleotide, an interfering RNA (e.g., a small interfering RNA (siRNA), a catalytic RNA, a micro RNA (miRNA), double-stranded RNA (dsRNA), a non-coding RNA (ncRNA), mitochondrial RNA (mtDNA) or a ribozyme.

In some embodiments, the therapeutic oligonucleotide can be selected from the group consisting of, but not limited to, fomivirsen, mipomersen, eteplirsen, nusinersen, AP 12009, AVI-6002, AVI-6003, Volanesorsen just to name a few.

In some embodiments, the macromolecule is a compound comprising a modified oligonucleotide of SEQ ID NO: 1.

In some embodiments, the polynucleotide encodes a peptide to be expressed in vivo in a subject.

In some embodiments, the macromolecule is a DNA molecule, e.g., a double stranded or single stranded DNA molecule. In some embodiments, the DNA encodes a peptide to be expressed in vivo in a subject. For example, in some embodiments, the DNA molecule is a viral vector.

The skilled artisan can appreciate that additional oligonucleotides can be used in the present disclosure, and the above list is for exemplary purposes only. In some embodiments, the oligonucleotides have a modified backbone to increase stability and half-life. Additionally, in some embodiments, oligonucleotides are conjugated to another molecule to improve delivery, e.g., a targeting protein, and/or stability of the oligonucleotides. Such modifications are envisioned by the present disclosure and the term “oligonucleotide” encompasses such modifications.

In some embodiments, the macromolecule is a protein, i.e., a peptide. In some embodiments, the peptide can be 5 to 3000 amino acids in length, 25 to 2000 amino acids in length, or 100 to 1000 amino acids in length. In other embodiments, the peptide can be 5 to 200 amino acids in length, such as 10 to 150 amino acids in length, for example, 20 to 100 amino acids in length, for instance, 30 to 75 amino acids in length, such as about 20 amino acids, about 30 amino acids, about 40 amino acids, about 50 amino acids, about 60 amino acids, about 70 amino acids, about 80 amino acids, about 90 amino acids, or about 100 amino acids in length.

In certain embodiments, the peptide is modified, for example, by lipid modification. In certain embodiments, the peptide is modified via lactamization, disulfide bridge closure, lipidation such as palmitoylation, and/or PEGylation. In certain embodiments, the peptide comprises at least one lapidated amino acid residue. In certain embodiments, the lipidated peptide comprises at least two lipidated amino acid residues. In certain embodiments, the lipidated peptide contains only one lipidated amino acid residue. As used herein, a peptide with one lipid or lipid moiety attached is referred to as a mono-lipidated peptide. In other embodiments, the lipidated peptide contains two lipidated amino acid residues. As used herein, a peptide with two lipids or lipid moieties attached is referred to as a bis-lipidated peptide. In certain embodiments, the lipidated peptide is a synthetic peptide. In certain embodiments, the lipidated peptide is a synthetic peptide. In certain embodiments, the lipidated synthetic peptide comprises at least one substitution of an alpha-methyl functionalized amino acid for a native amino acid residue. In other embodiments, a lipidated synthetic peptide comprises at least two, three, four, five, six, or more substitutions of alpha-methyl functionalized amino acids for native amino acid residues.

In certain embodiments, the lipidated peptide described herein is derived from the sequence of GLP-1 and referred to herein as a lipidated GLP-1 peptide analog. Sequences for the native (wild type) peptides of the various peptides and classes of peptides described herein that can be prepared to yield synthetic peptides having the recited characteristics are well known in the art. The native amino acid sequence for GLP-1 (7-36) is known in the art as set forth below: HAEGTFTSDVSSYLEGQAAKEFIAWLVKGR (SEQ ID NO: 2) (Table 1).

In certain embodiments, the peptide comprises the amino acid sequence HAibEGS-(αMeF)TSDV-(αMeS)SX13LE-GEAA(αMeK)-E(αMeF)IAX25-VVEGG
wherein: X13 is a lipid modified K; X25 is a lipid modified K; and where “αM” is an alpha-methyl functionalized amino acid, and wherein the two lipid modified K residues are the same or are different, and are selected from the group consisting of: K(ε-(PEG)₂-(PEG)₂-γE-Lauroyl), K(ε-(PEG)₂-(PEG)₂-γE-Myristoyl), K(ε-(PEG)₂-(PEG)₂-γE-Palmitoyl), K(ε-(PEG)₂-(PEG)₂-γE-Stearoyl), and any combination thereof, such as described in International Patent Application No. PCT/EP16/063206.

In certain embodiments, the peptide comprises a C-terminal amide. In certain embodiments, the peptide comprises a C-terminal acid.

In certain embodiments, the polypeptide comprises the amino acid sequence of SEQ ID NO: 3 (Table 1), SEQ ID NO: 4 (Table 1), SEQ ID NO: 5 (Table 1), or SEQ ID NO: 6 (Table 1):

TABLE 1
Peptide Sequences

| | SEQ ID NO | |
|---|---|---|
| GLP-1 (7-36) | 2 | HAEGT⁵ FTSDV¹⁰ SSYLE¹⁵ GQAAK²⁰ EFIAW²⁵ LVKGR³⁰-amide |
| | 3 | H-(Aib)²-EG-(S)⁵-(α-MeF)⁶-TSDV¹⁰-(α-MeS)¹¹-S-K(ϵ-(PEG)₂-(PEG)₂-γE- |
| | | Lauroyl)¹³-LE¹⁵ G-(E)¹⁷-AA-(α-MeK)²⁰E-(α-MeF)²²IA-K(ϵ-(PEG)₂- |
| | | (PEG)₂-γE-Lauroyl)²⁵-(V)₂₆-V-(E)²⁸-G-(G)³⁰ |
| | 4 | H-(Aib)²-EG-(S)⁵-(α-MeF)⁶-TSDV¹⁰-(α-MeS)¹¹-S-K(ϵ-(PEG)₂-(PEG)₂-yϵ- |
| | | Myristoyl)¹³-LE¹⁵G-(E)¹⁷-AA-(α-MeK)²⁰E-(α-MeF)²²IA-K(ϵ-(PEG)₂- |
| | | (PEG)₂-yE-Myristoyl)²⁵-(V)²⁶-V-(E)²⁸-G-(G)³⁰ |
| | 5 | H-(Aib)²-EG-(S)⁵-(α-MeF)⁶-TSDV¹⁰-(α-MeS)¹¹-S-K(ϵ-(PEG)₂-(PEG)₂-Yϵ- |
| | | Palmitoyl)¹³-LE¹⁵G-(E)¹⁷-AA-(α-MeK)²⁰E-(α-MeF)²²IA-K(ϵ-(PEG)₂- |
| | | (PEG)₂-γE-Palmitoyl)²⁵-(V)²⁶-V-(E)²⁸-G-(G)³⁰ |
| | 6 | H-(Aib)²-EG-(S)⁵-(α-MeF)⁶-TSDV¹⁰-(α-MeS)¹¹-S-K(ϵ-(PEG)₂-(PEG)₂-γE- |
| | | Stearoyl)¹³-LE¹⁵G-(E)¹⁷-AA-(α-MeK)²⁰E-(α-MeF)²²IA-K(ϵ-(PEG)₂- |
| | | (PEG)₂-γE-Stearoyl)²⁵-(V)²⁶-V-(E)²⁸-G-(G)³⁰ |

Various therapeutic proteins are known in the art and can be suitable to be used with the disclosure herein. In some embodiments, the therapeutic protein can be selected from the group consisting of, but not limited to, an antigen, an antibody or antibody fragment, an enzyme, a cytokine, a therapeutic protein, a chemokine, a regulatory protein, a structural protein, a chimeric protein, a nuclear protein, a transcription factor, a viral protein, a TLR protein, an interferon regulatory factor, an angiostatic or angiogenic protein, an apoptotic protein, an Fc gamma receptor, a hematopoietic protein, a tumor suppressor, a cytokine receptor, or a chemokine receptor. In some embodiments, the protein is an enzyme or hormone. In some embodiments, the protein is a clotting factor, a growth factor or a cytokine.

Representative antigens include, without limitation: those derived from Cholera toxoid, tetanus toxoid, diphtheria toxoid, hepatitis B surface antigen, hemagglutinin, neuraminidase, influenza M protein, PfHRP2, pLDH, aldolase, MSP1, MSP2, AMA1, Der-p-1, Der-f-1, Adipophilin, AFP, AIM-2, ART-4, BAGE, alpha-fetoprotein, BCL-2, Bcr-Abl, BING-4, CEA, CPSF, CT, cyclin DIEp-CAM, EphA2, EphA3, ELF-2, FGF-5, G250, Gonadotropin Releasing Hormone, HER-2, intestinal carboxyl esterase (iCE), IL13Ralpha2, MAGE-1, MAGE-2, MAGE-3, MART-1, MART-2, M-CSF, MDM-2, MMP-2, MUC-1, NY-EOS-1, MUM-1, MUM-2, MUM-3, p53, PBF, PRAME, PSA, PSMA, RAGE-1, RNF43, RU1, RU2AS, SART-1, SART-2, SART-3, SAGE-1, SCRN 1, SOX2, SOX10, STEAP1, surviving, Telomerase, TGFbetaRII, TRAG-3, TRP-1, TRP-2, TERT, or WT1; those derived from a virus, such as Cowpoxvirus, Vaccinia virus, Pseudocowpox virus, Human herpesvirus 1, Human herpesvirus 2, Cytomegalovirus, Human adenovirus A-F, Polyomavirus, Human papillomavirus, Parvovirus, Hepatitis A virus, Hepatitis B virus, Hepatitis C virus, Human immunodeficiency virus, Orthoreovirus, Rotavirus, Ebolavirus, parainfluenza virus, influenza A virus, influenza B virus, influenza C virus, Measles virus, Mumps virus, Rubella virus, Pneumovirus, Human respiratory syncytial virus, Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella; those derived from a bacterium, such as Anthrax, Brucella, Candida, Chlamydia pneumoniae, Chlamydia psittaci, Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica; or those derived from a protozoa, e.g. Plasmodium falciparum.

Various means of modifying a protein are known in the art to increase (or in some instances decrease) its stability and/or half-life. Likewise, methods of modifying the protein to increase its targeting to increase its effectiveness as know. In some embodiments, the protein is post-translationally modified. In some embodiments, the post-translational modification is glycosylation or phosphorylation. Such modifications are envisioned by the present disclosure and the term “oligonucleotide” encompasses such modifications.

In some embodiments, the macromolecule is a therapeutic macromolecule suitable for the treatment of a disease or disorder.

Sodium caprate, also known as sodium decanoate, is a plant-derived compound with the chemical formula C₁₀H₁₉NaO₂ and the following structure:

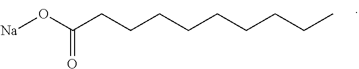

The present disclosure discloses that that sodium caprate has more than one solid form. A compound may have more than one solid form due to variations in the crystal lattice or structure; molecular packing; and/or amount of solvent in the packed molecules; and/or combination of them. Several distinct forms of sodium caprate have been identified, including Form A, Form B, Form C, and Form D. Methods of identifying the sodium caprate forms are described herein and include various analytical methods, such as X-ray diffraction, X-ray scattering, and water content determination by Karl-Fischer titration.

In some embodiments, a sodium caprate form is identified by small-angle X-ray scattering (SAXS). In some embodiments, a sodium caprate form is identified by wide-angle X-ray scattering (WAXS). SAXS and WAXS are scattering techniques in which X-rays are scattered by fluctuations in the electron density in the sample. Thus, in some embodiments, SAXS and WAXS are used to determine the crystalline structure. SAXS typically diffracts at a smaller angle than WAXS (i.e., the distance between the sample and detector is longer for SAXS than WAXS). Methods of preparing a SAXS or WAXS experimental set-up are known to the skilled artisan.

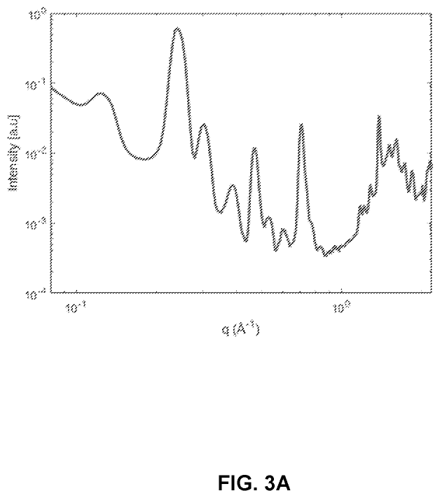

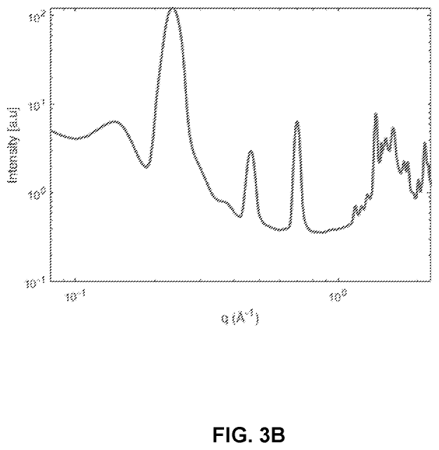

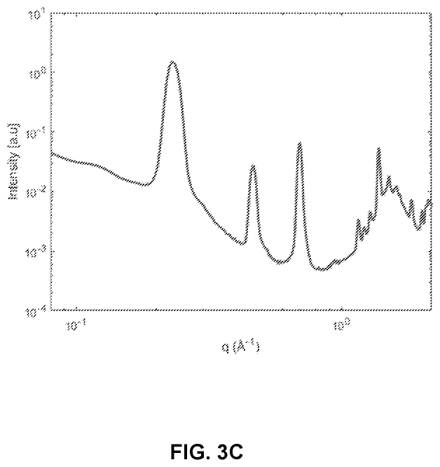

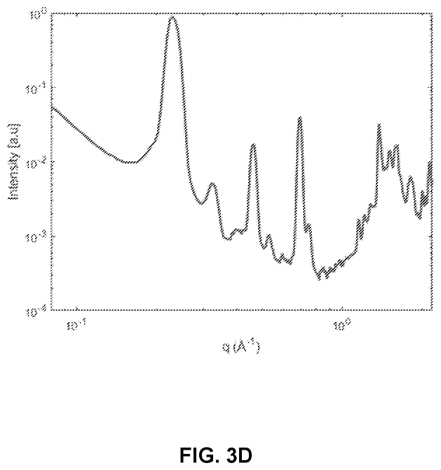

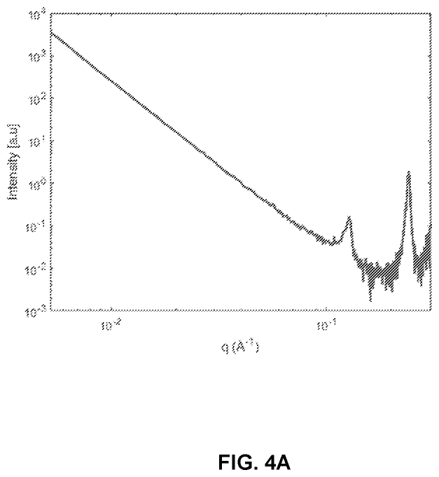

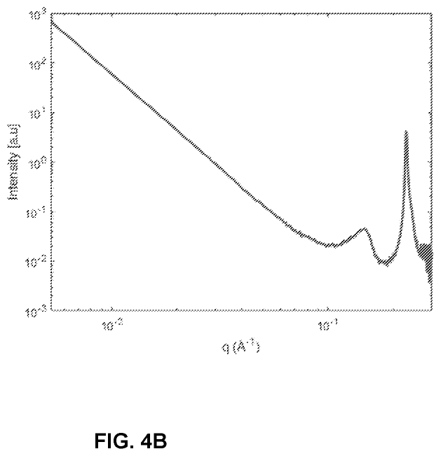

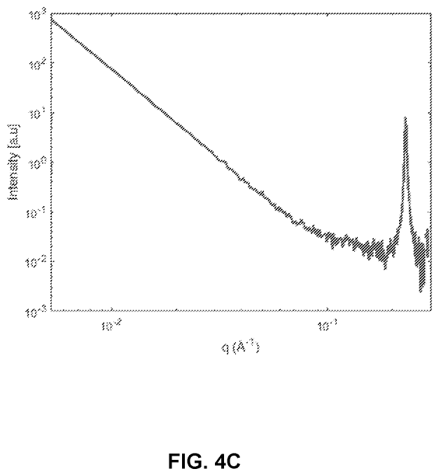

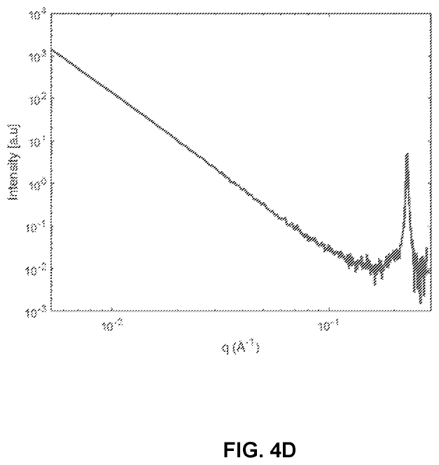

In some embodiments, the sodium caprate Form A, B, C and D as described herein has a WAXS or SAXS pattern substantially as shown in the FIGS. 3A-3D and 4A-4D. The term “substantially as shown in” when referring, for example, to an WAXS or SAXS pattern, refers to a pattern that is not necessarily identical to those depicted herein, but that falls within the limits of experimental error or deviations when considered by one of ordinary skill in the art. Various values for WAXS and SAXS are described herein. As used throughout the present disclosure (unless explicitly noted), all WAXS and SAXS peak position values are to be construed to be ±0.02. For simplicity, this variance is not explicitly noted next to each peak value. For example, if the disclosure indicates Form A has a peak at 0.12 Å⁻¹, this includes 0.12±0.02 Å⁻¹, i.e., 0.10 Å⁻¹ to 0.14 Å⁻¹.

In some embodiments, SAXS and/or WAXS is used to determine the crystalline structure of a compound. In some embodiments, Form A sodium caprate comprises a WAXS peak at a region of about 0.1 to about 0.15 Å⁻¹. In some embodiments, Form A sodium caprate comprises more than one WAXS peaks at a region of about 0.12 to about 0.23 Å⁻¹. In some embodiments, Form A sodium caprate comprises a WAXS peak at about 0.12 Å⁻¹. In some embodiments, Form A sodium caprate comprises a WAXS peak at about 0.23 Å⁻¹.

In some embodiments, Form B sodium caprate comprises a WAXS peak at a region of about 0.1 to about 0.2 Å⁻¹. In some embodiments, Form B sodium caprate comprises a WAXS peak at about 0.17 Å⁻¹. In some embodiments, Form B sodium caprate comprises a WAXS peak at a region of about 0.23 Å⁻¹.

In some embodiments, Forms C and D sodium caprate comprise only one peak at a region of about 0.1 to about 0.3 Å⁻¹. In some embodiments, Form C and D do not comprise a WAXS peak at a region of about 0.1 to about 0.2 Å⁻¹. In some embodiments, Forms C and D sodium caprate do not comprise WAXS peaks at about 0.12 Å⁻¹ and 0.17 Å⁻¹. In some embodiments, Form C and D comprise a WAXS peak at about 0.23 Å⁻¹.

In some embodiments, Form A sodium caprate comprises a SAXS peak at a region of about 0.1 to about 0.15 Å⁻¹. In some embodiments, Form A sodium caprate comprises more than one SAXS peaks at a region of about 0.12 to about 0.23 Å⁻¹. In some embodiments, Form A sodium caprate comprises a SAXS peak at about 0.12 Å⁻¹. In some embodiments, Form A sodium caprate comprises a SAXS peak at about 0.23 Å⁻¹.

In some embodiments, Form B sodium caprate comprises a SAXS peak at a region of about 0.1 to about 0.2 Å⁻¹. In some embodiments, Form B sodium caprate comprises a SAXS peak at about 0.17 Å⁻¹. In some embodiments, Form B sodium caprate comprises a SAXS peak at a region of about 0.23 Å⁻¹.

In some embodiments, Forms C and D sodium caprate comprise only one SAXS peak at a region of about 0.1 Å⁻¹ to about 0.3 Å⁻¹. In some embodiments, Forms C and D sodium caprate do not comprise a SAXS peak at 0.12 Å⁻¹.

In some embodiments, a sodium caprate form is identified by X-ray powder diffraction (XRPD). XRPD is a diffraction method, i.e., scattering from atoms in planes in an ordered crystal lattice. In general, XRPD can be used to detect unique fingerprints of crystallographic unit cells present within a crystalline substance, with each type of unit cell appearing as a peak in a particular position on an XRPD pattern. Thus, crystalline substances may be distinguished by their unit cells via identification of the peaks appearing on the diffraction pattern. Methods of preparing a XRPD experimental set-up are known to the skilled artisan.

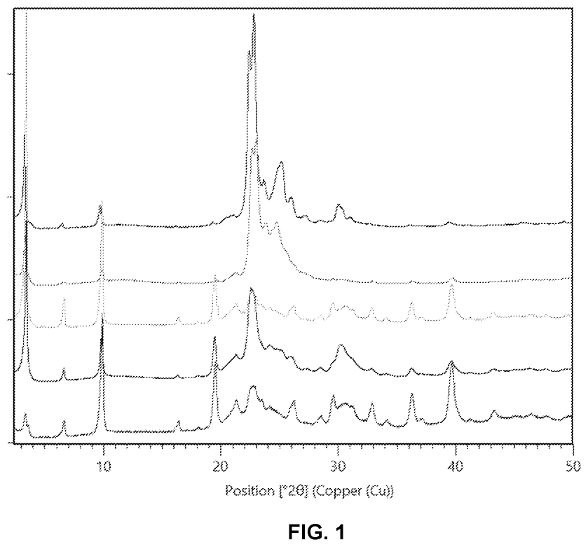

In some embodiments, the sodium caprate as described herein has a XRPD pattern substantially as shown in the FIGS. 1 and/or 2A-2D. The term “substantially as shown in” when referring, for example, to an XRPD pattern, refers to a pattern that is not necessarily identical to those depicted herein, but that falls within the limits of experimental error or deviations when considered by one of ordinary skill in the art. Various values for XRPD are described herein. As used throughout the present disclosure (unless explicitly noted), all XRPD peak position values are to be construed to be ±0.5° 2θ.

In some embodiments, Form A sodium caprate comprises a XRPD peak at about 4° 2θ. In some embodiments. In some embodiments, Forms B, C or D sodium caprate do not comprise a XRPD peak at about 4° 2θ.

In some embodiments, the water content of sodium caprate is determined by Karl Fischer titration. Karl Fischer titration uses coulometric or volumetric titration to determine trace amounts of water in a sample. Methods of performing Karl Fischer titration are known to the skilled artisan. In some embodiments, Form A or Form B sodium caprate has a lower water content compared with Form C or Form D water content. In some embodiments, the sodium caprate is Form A sodium caprate characterized by a water content by Karl Fischer titration below 2%, below 1.9%, below 1.8%, below 1.7%, below 1.6%, below 1.5%, or below 1.4%. In some embodiments, Form A sodium caprate has a water content of about 0.4% to about 1.7%. Form B sodium caprate has a water content ≤1%. Form C sodium caprate has a water content of about 2% to about 3%, and Form D sodium caprate has a water content of about 2.5% to about 4%. In some embodiments, the sodium caprate used for making the oral tablet has a water content of less than about 2% as measured by Karl Fischer titration. In some embodiments, the sodium caprate has a water content of less than about 1.5%. In some embodiments, the sodium caprate has a water content of about 1%, about 1.1%, about 1.2%, about 1.3%, about 1.4%, about 1.5%, about 1.6%, about 1.7%, about 1.8%, about 1.9%, or about 2.0%.

In some embodiments, the sodium caprate has a specific particle size range prior to mixing with the macromolecule. The present disclosure has found that varying particle size of the sodium caprate can alter tablet tensile strength and friability. In some embodiments, varying particle size can affect the amount of mixture that sticks to the equipment during the tableting process, e.g., the mixture comprising sodium caprate and the macromolecule sticks or leaves residue on the tableting die and press. In some embodiments, the sodium caprate is milled to achieve the desired particle size. In some embodiments, the sodium caprate is sieved to achieve the desired particle size. In some embodiments, the sodium caprate is sieved or milled to obtain a desired particle size before the direct compression to form a tablet. In some embodiments, the mixture comprising sodium caprate and the macromolecule is sieved or milled to obtain a desired particle size before the direct compression to form a tablet. Thus, in some embodiments, the disclosure provides for a method of reducing the sticking of the mixture during the tableting process by providing a specific particle size of sodium caprate.

In some embodiments, the sodium caprate has a particle size of about 35 μm to about 1300 μm. In some embodiments, the sodium caprate has a particle size of about 100 μm to about 850 μm. In some embodiments, the sodium caprate has a particle size of about 250 μm to about 700 μm.

In some embodiments, the method of making the oral tablet provided by the present disclosure comprises forming a coating on the tablet after compression. Various coatings and processes for applying the coat are known in the art. In some embodiments, the coating can mask an odor color or taste of the table, can offer physical and/or chemical protection to the drug, control and/or sustain release of the drug, increase the mechanical strength of the tablet, or protect the drug from the gastric environment.

The disclosure as described herein provides a method for making an oral tablet which does not require wet granulation. Accordingly, in some embodiments, the disclosure as described herein provides a method for making an oral tablet which does not comprise a drying step after the mixing of the macromolecule with sodium caprate. In some embodiments, the disclosure as described herein provides a method for making an oral tablet which does not comprise a milling step after the wet granulation process. Thus, the mixture of macromolecule and sodium caprate of the present disclosure can be directly compressed into tablets without the need for wet granulation, drying, and/or additional milling step resulting in a simplified tablet manufacturing process. The simplified process can result in greater efficiencies, including reduced operating costs, reduced loss or products, increased manufacturing rate, and decreased use of specialized equipment required to manufacture the tablets. In some embodiments, tablets made by the processes lacking wet granulation have a decrease in stickiness of the mixture comprising the macromolecule and sodium caprate to the tableting equipment.

The tablets as described in the disclosure can be made by compressing the mixture comprising sodium caprate and the macromolecule. In some embodiments, the compression is performed at greater than 10 MPa. In some embodiments, the compression is performed at 15 MPa to 500 MPa. In some embodiments, the compression is performed at 25 MP to 300 MPa. In some embodiments, the compression c is performed at 50 MP to 200 MPa. In some embodiments, the disclosure has found that compressing below 50 MPa results in an increase in sticking of the mixture to the tableting press.

Sodium caprate, as provided herein, can be used as a permeation enhancer to increase the uptake of the macromolecule by the subject. In some embodiments, the amount of sodium caprate is maximized in a tablet. In some embodiments, the sodium caprate is about 30% to about 99% by weight of the tablet. In some embodiments, the sodium caprate is about 50% to about 85% or about 60% to about 80% by weight of the tablet. In some embodiments, the sodium caprate is about 50%, about 55%, about 60%, about 65%, about 70%, about 75%, about 80%, about 85%, or about 90% by weight of the tablet. In some embodiments, the sodium caprate is greater than 80%, greater than 85%, greater than 90% or greater than 95% by weight of the tablet. For examiner, in some embodiments, the sodium caprate is about 90%, about 92%, about 94%, or about 96% by weight of the tablet.

Various excipients are known in the art and can be included in the tablets as described herein. Excipients can include, but are not limited to a carrier, excipient, glidant, sweetening agent, diluent, preservative, dye/colorant, flavor enhancer, surfactant, wetting agent, dispersing agent, suspending agent, stabilizer, isotonic agent, solvent, and/or emulsifier, or combinations there. In some embodiments, the excipient is pharmaceutically acceptable. “Pharmaceutically acceptable” includes compounds which have been approved by the United States Food and Drug Administration as being acceptable for use in humans or domestic animals. In some embodiments, the mixture of macromolecule and sodium caprate further comprises a polyol. In some embodiments, the polyol is sorbitol, mannitol, maltitol, xylitol or combinations thereof. In some embodiments, the polyol is mannitol.

Various amounts of excipients can be used according to the present disclosure. In some embodiments, the excipient is about 0.001% to about 50% by weight of the tablet. In some embodiments, the excipient is about 0.01% % to about 45% or about 0.1% to about 40% by weight of the tablet. In some embodiments, the excipient is about 1%, about 5%, about 10%, about 20%, about 30%, about 40% or about 50% by weight of the tablet. In some embodiments, the excipient is less than 10%, less than 5%, less than 20% or less than 1% by weight of the tablet. For example, in some embodiments, the excipient is about 0.1%, about 0.5%, about 1%, or about 2% by weight of the tablet. In some embodiments, no excipients (with the exception of sodium caprate) are in the tablet.

Various amounts of polyol can be used according to the present disclosure. In some embodiments, the polyol is about 0.001% to about 50% by weight of the tablet. In some embodiments, the polyol is about 0.01% % to about 45% or about 0.1% to about 40% by weight of the tablet. In some embodiments, the polyol is about 1%, about 5%, about 10%, about 20%, about 30%, about 40% or about 50% by weight of the tablet. In some embodiments, the polyol is less than 10%, less than 5%, less than 20% or less than 1% by weight of the tablet. For example, in some embodiments, the polyol is about 0.1%, about 0.5%, about 1%, or about 2% by weight of the tablet. In some embodiments, no polyol (with the exception of sodium caprate) are in the tablet.

The methods as provided herein can provide a tablet comprising an effective amount of a therapeutic macromolecule. “Effective amount” or “therapeutically effective amount” refers to an amount of a macromolecule, which when administered to a patient in need thereof, is sufficient to effect treatment for disease-states, conditions, or disorders for which the macromolecule has utility. Such an amount would be sufficient to elicit the biological or medical response of a tissue system, or patient that is sought by a researcher or clinician. The amount of a macromolecule according to the disclosure which constitutes a therapeutically effective amount will vary depending on such factors as the macromolecule and its biological activity, the formulation used for administration, the time of administration, the half-life of the macromolecule, the rate of excretion of the macromolecule, the duration of the treatment, the type of disease-state or disorder being treated and its severity, drugs used in combination with or coincidentally with the macromolecule of the disclosure, and the age, body weight, general health, sex and diet of the patient. Such a therapeutically effective amount can be determined routinely by one of ordinary skill in the art having regard to their own knowledge, the state of the art, and this disclosure. In some embodiments, the macromolecule is about 0.1% to about 12% by weight of the tablet, about 0.2% to about 10% by weight of the tablet, about 0.3% to about 8% by weight of the tablet, about 0.4% to about 5% by weight of the tablet, or about 0.5% to about 4% by weight of the tablet.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The tablets according to the present disclosure are suitable for oral administration to a subject or patient. The terms “subject” or “patient” refer to an animal, such as a mammal (including a human), that has been or will be the object of treatment, observation or experiment. The methods described herein may be useful in human therapy and/or veterinary applications. In some embodiments, the subject is a mammal (or the patient). In some embodiments the subject (or the patient) is human, domestic animals (e.g., dogs and cats), farm animals (e.g., cattle, horses, sheep, goats and pigs), and/or laboratory animals (e.g., mice, rats, hamsters, guinea pigs, pigs, rabbits, dogs, and monkeys). In some embodiments, the subject (or the patient) is a human. “Human (or patient) in need thereof” refers to a human who may have or is suspect to have diseases or conditions that would benefit from certain treatment; for example, being treated with the compounds disclosed herein according to the present application.

The tablets described herein include solid compressed dosage forms of a size and shape suitable for oral administration, e.g., a caplet would be embodied by the term “tablet” as described herein. In some embodiments, the tablet is from 5 mm in diameter to 25 mm in diameter, or 8 mm to 20 mm in diameter. In some embodiments, the tablet is circular in shape. In some embodiments, the tablet is oval in shape. In some embodiments, the tablet is elongated, e.g., shaped like a capsule. The tablets may, where appropriate, be conveniently presented in discrete dosage units and may be prepared by any of the methods well known in the art of pharmacy.

Compressed tablets may be prepared by compressing in a suitable machine an active compound in a form such as a powder or granules optionally mixed with sodium caprate as described herein, and optionally further comprising an excipient, e.g., a lubricant, inert diluent, lubricating agent, stabilizer, emulsifier, disintegrant, surface-active agent or dispersing agent, etc. In some embodiments, the tablets can also comprise a coating.

The tablets of the present disclosure may be prepared using pharmaceutical processes namely by direct compression or by granulation processing and final tableting. The process may comprise the steps of initially forming a core comprising the active agent and subsequently surrounding core with one or more layers.

The tensile strength of the tablet of the present disclosure (measurement with a tablet hardness tester) must be sufficient for storage and administration of table. In some embodiments, the tablet has a tablet hardness tensile strength of above 0.5 MPa. In some embodiments, the oral tablet has a tablet hardness tensile strength ranging from about 0.5 MPa to about 5 MPa, for example, ranging from about 0.75 MPa to about 3.0 MPa.

In some embodiments, the disclosure is directed to a method of making an oral tablet, the method comprising: a) sieving sodium caprate to obtain sodium caprate particles having a particle size of about 35 μm to about 1100 μm, b) mixing the sodium caprate particles with a macromolecule to form a mixture, and c) direct compressing the mixture of to form a tablet, wherein the sodium caprate is Form A sodium caprate characterized by a wide-angle X-ray scattering (WAXS) spectrum which includes a peak at a region of 0.1 to 0.15 Å⁻¹, which includes a peak at 0.12 and 0.23 Å⁻¹, and wherein the method does not comprise a wet granulation step, a drying step or a milling step after the mixing of step (a).

In some embodiments, the disclosure is directed to a method of making an oral tablet, where the conversion of any sodium caprate Forms to sodium caprate Form A is achieved by introducing a wet granulation step of sodium caprate with a non-aqueous solvent in the tablet manufacturing process, wherein the Form A sodium caprate is characterized by a X-ray powder diffraction (XRPD) spectrum which includes a peak at 4° 2θ and/or by a wide-angle X-ray scattering (WAXS) spectrum which includes a peak at a region of 0.1 to 0.15 Å⁻¹ and with peaks at 0.12 and 0.23 Å⁻¹

In some embodiments, the disclosure is directed to a method of making an oral tablet, the method comprising: a) mixing sodium caprate particles with additional excipients to form a mixture; b) granulating the mixture with an organic non-aqueous solvent to form granules; c) drying the granules; d) sieving or milling the granules to obtain granules having a particle size of about 100 μm to 2000 μm; e) blending with a macromolecule and/or additional excipients; f) compressing the mixture to form a tablet, wherein the sodium caprate after step c) is Form A sodium caprate. In some embodiments, Form A sodium caprate is characterized by an X-ray powder diffraction (XRPD) spectrum which includes a peak at 4° 2θ. In some embodiments, the organic solvent is chosen from methanol (MeOH), ethanol (EtOH), 2-propanol (IPA), toluene, cyclohexane, dioxane, dimethylformamide (DMF), acetonitrile (ACN), tetrahydrofuran (THF), methyl isobutyl ketone (MIBK), ethyl acetate (EtOAc), and diethylether. In at least one embodiment, the organic solvent is ethanol. In some embodiments, the additional excipient is a polyol. In some embodiments, the polyol is mannitol.

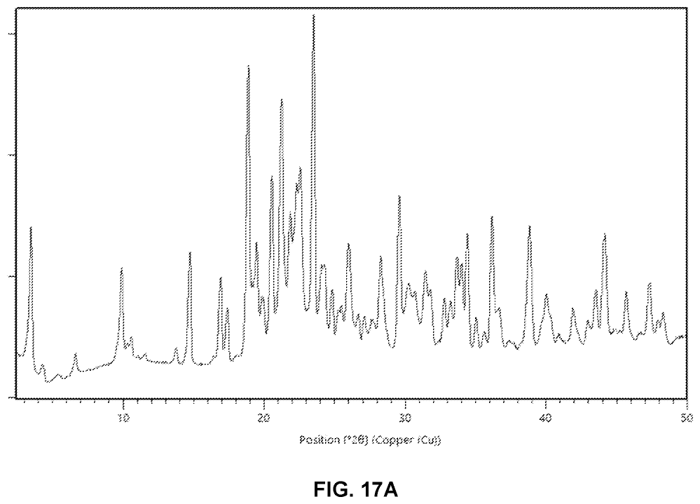

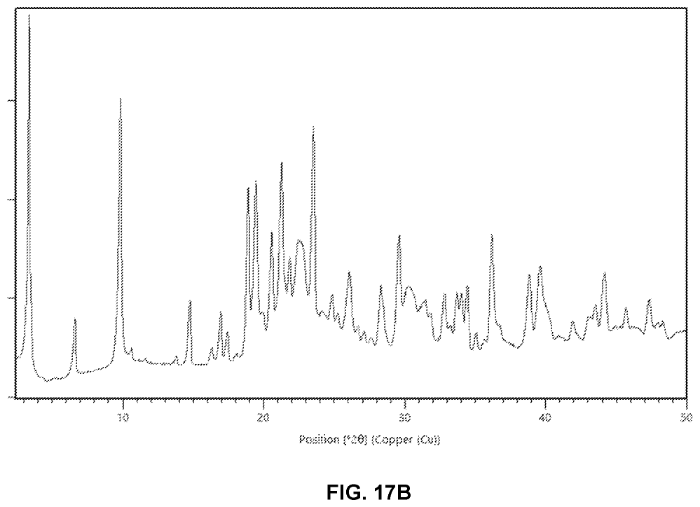

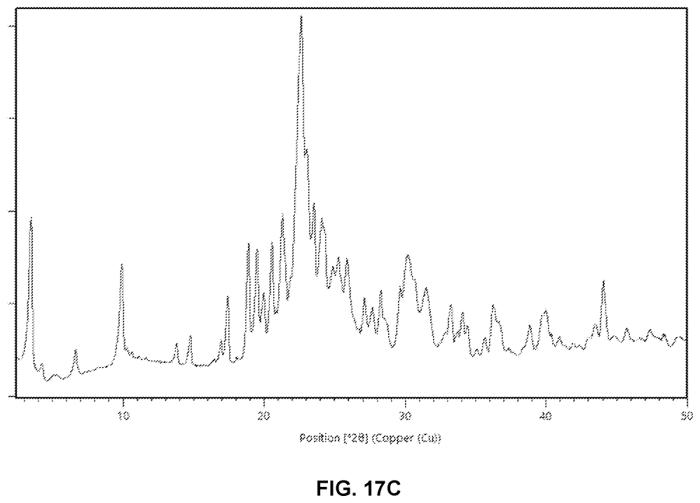

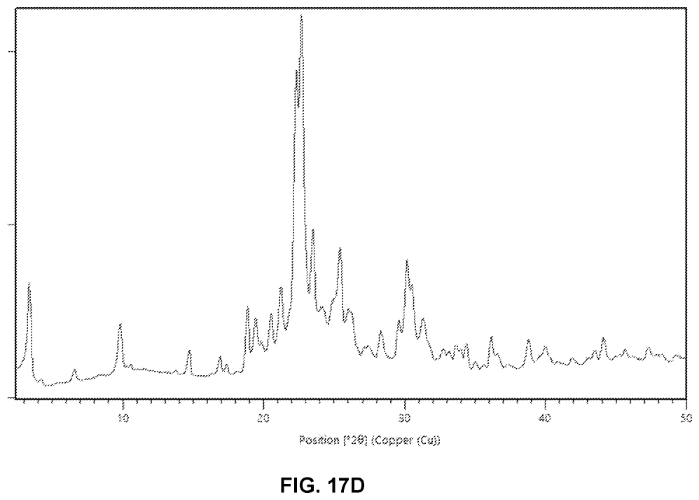

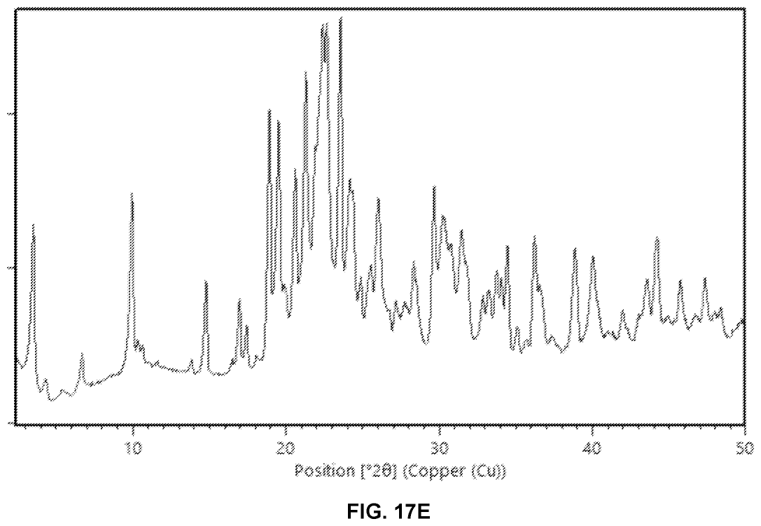

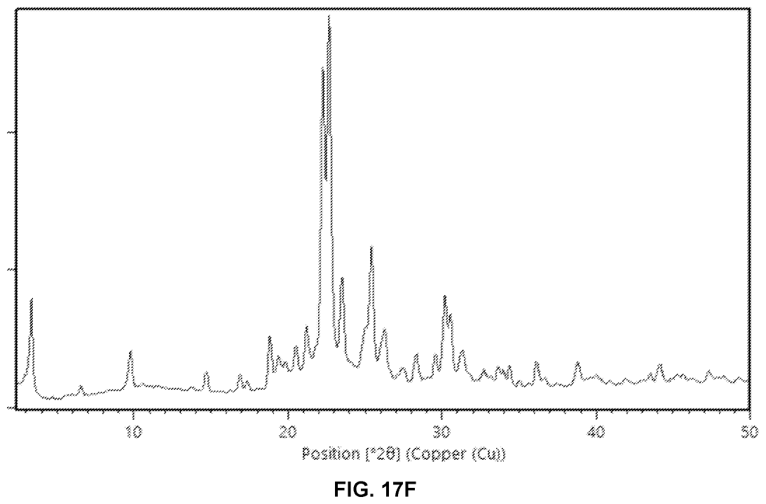

In some embodiments, the disclosure is directed to a method of making an oral tablet, the method comprising: a) granulating sodium caprate particles with an organic non-aqueous solvent to form granules; b) drying the granules; c) sieving or milling the granules to obtain granules having a particle size of about 100 μm to 2000 μm; and d) blending the granules with a macromolecule and/or additional excipients to form a mixture; e) compressing the mixture to form a tablet wherein the sodium caprate after step (b) is Form A sodium caprate. In some embodiments, Form A sodium caprate is characterized by a X-ray powder diffraction (XRPD) spectrum which includes a peak at 4° 2θ and/or by a wide-angle X-ray scattering (WAXS) spectrum which includes a peak at a region of 0.1 to 0.15 Å⁻¹ and with peaks at 0.12 and 0.23 Å⁻¹. In some embodiments, the organic solvent is chosen from methanol (MeOH), ethanol (EtOH), 2-propanol (IPA), toluene, cyclohexane, dioxane, dimethylformamide (DMF), acetonitrile (ACN), tetrahydrofuran (THF), methyl isobutyl ketone (MIBK), ethyl acetate (EtOAc), and diethylether. In at least one embodiment, the organic solvent is ethanol. In some embodiments, the additional excipient is a polyol. In some embodiments, the polyol is mannitol. An exemplary process for tablet production that include wet granulation for sodium caprate, and in which neither the macromolecule nor the other excipients are subject to wet granulation and drying is illustrated at FIG. 17.

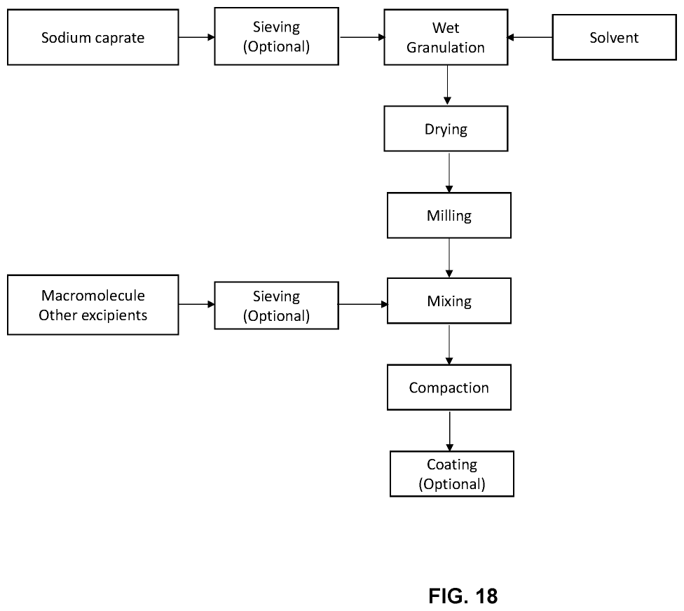

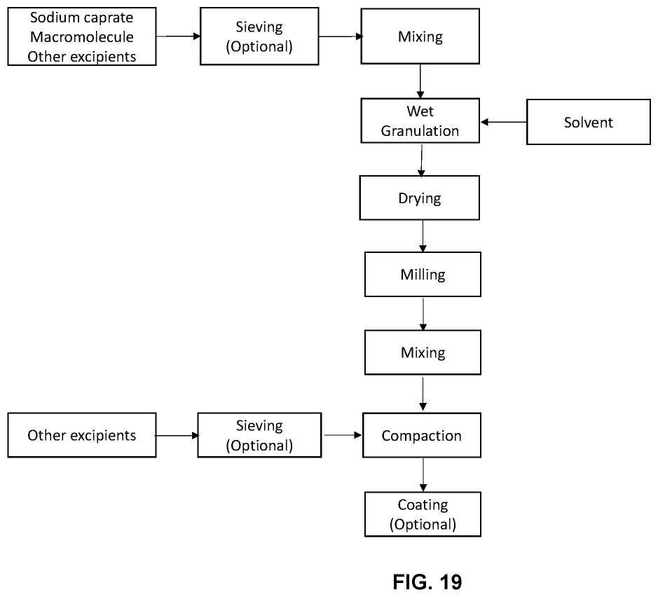

In some embodiments, the disclosure is directed to a method of making an oral tablet, the method comprising: a) mixing sodium caprate particles with additional excipients and a macromolecule to form a mixture; b) granulating the mixture with an organic non-aqueous solvent to form granules; c) drying the granules; d) sieving or milling the granules to obtain granules having a particle size of about 100 μm to 2000 μm; e) blending the granules with additional excipients to form a mixture; f) compressing the mixture to form a tablet, wherein the sodium caprate after step c) is Form A sodium caprate. In some embodiments, sodium caprate is characterized by an X-ray powder diffraction (XRPD) spectrum which includes a peak at 4° 2θ. In some embodiments, the organic non-aqueous solvent is chosen from methanol (MeOH), ethanol (EtOH), 2-propanol (IPA), toluene, cyclohexane, dioxane, dimethylformamide (DMF), acetonitrile (ACN), tetrahydrofuran (THF), methyl isobutyl ketone (MIBK), ethyl acetate (EtOAc), and diethylether and isopropyl alcohol (IPOH). In at least one embodiment, the organic non-aqueous solvent is ethanol. In at least one embodiment, the organic non-aqueous solvent is isopropyl alcohol. An exemplary process for tablet production that include wet granulation of sodium caprate together with the macromolecule and other excipients is illustrated at FIG. 18.

In some embodiments, the disclosure is directed to a tablet made by the methods described herein.

In some embodiments, the composition further comprises a polyol. In some embodiments, the polyol is mannitol. In some embodiments, the macromolecule is about 0.1% to about 12% by weight of the tablet. In some embodiments, the sodium caprate is about 45% to about 90% by weight of the tablet. In some embodiments, the polyol is about 0.001% to about 50% by weight of the tablet.

In some embodiments, the macromolecule is a peptide or an oligonucleotide. In some embodiments, the macromolecule is an antisense oligonucleotide. In some embodiments, the composition comprises an oligonucleotide of SEQ ID NO: 1. In some embodiments, the composition comprises a peptide of SEQ ID NOS: 3-6.

All references cited herein, including patents, patent applications, papers, textbooks and the like, and the references cited therein, to the extent that they are not already, are hereby incorporated herein by reference in their entirety.

### EXAMPLES

### Example 1. X-Ray Powder Diffraction Analysis of Sodium Caprate Forms

A preliminary X-ray powder diffraction (XRPD) experiment was used to screen sodium caprate from five different suppliers. Results in FIG. 1 show that sodium caprate from all five suppliers had different XRPD patterns. The differences in the diffraction patterns may be attributed to different forms of sodium caprate and/or to different mixtures of different sodium caprate forms.

Different forms of sodium caprate were prepared according to Table 1 and measured by XRPD.

TABLE 1
Sodium Caprate Preparation

| Form | Preparation |
|---|---|
| Form A | Slurry in ethanol, 50° C. overnight or slurry |
| | in acetonitrile, 5-65-5° C. temperature cycle |
| | for 2 hours in each step or heat at 140° C. |
| | for 10 mins, afterwards slurry in toluene overnight. |
| | Material subsequently dried. |
| Form B | Heat at 190-265° C. for approximately 3 hours |
| | and cool it to room temperature. |
| Form C | Dissolve in water and add acetone as anti-solvent. |
| | Material subsequently dried. |
| Form D | Slurry in THF:water (82:18% v/v), 3 days. |
| | Material subsequently dried. |

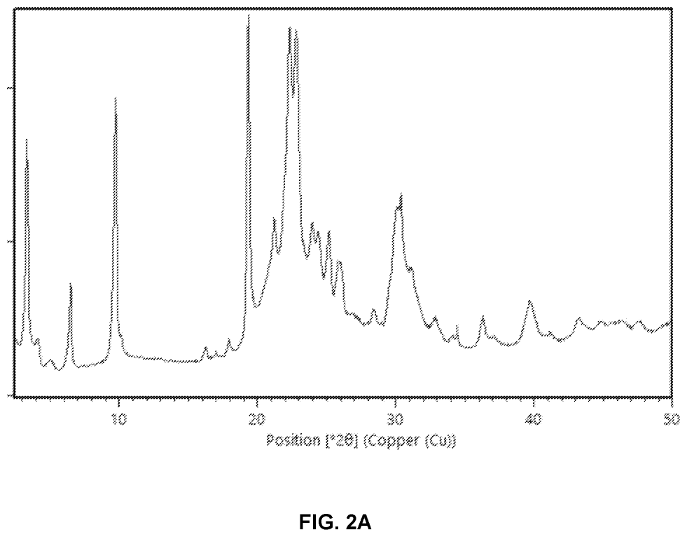

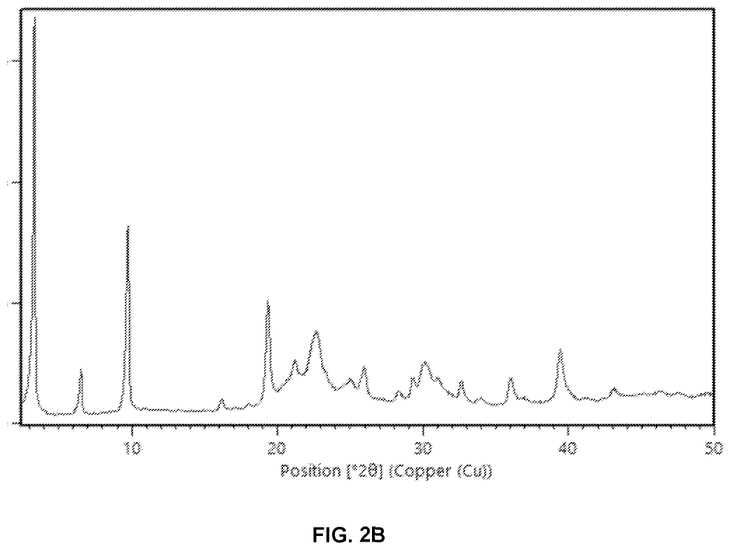

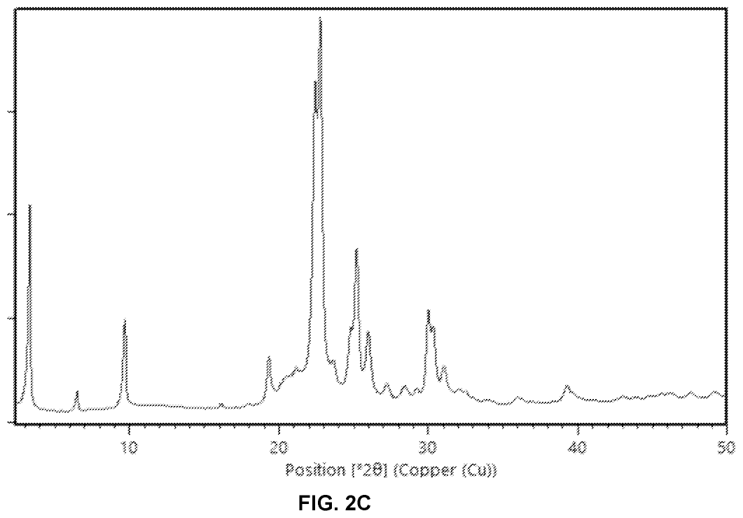

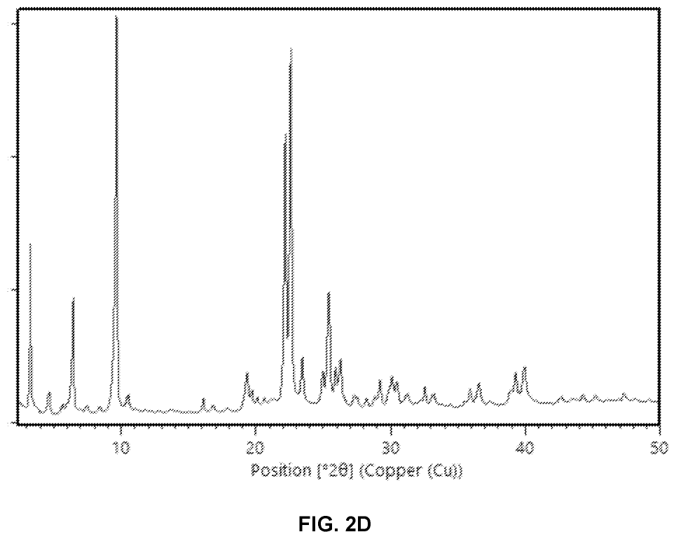

FIGS. 2A-2D show XRPD spectra of the different sodium caprate forms described in Table 1.

### Example 2. Wide-Angle X-Ray Scattering of Sodium Caprate Forms

Wide-angle x-ray scattering (WAXS) was used to further characterize the different forms of sodium caprate. Results are shown in FIGS. 3A-3D. Sodium caprate Forms A comprises more than one WAXS peaks at a region of about 0.12 to about 0.23 Å⁻¹. Sodium caprate Form A comprises a WAXS peak at about 0.12 Å⁻¹. Sodium caprate Form A comprises a WAXS peak at about 0.23 Å⁻¹. Sodium caprate Form B comprises a WAXS peak at a region of about 0.1 to about 0.2 Å⁻¹. Sodium caprate Form B comprises a WAXS peak at about 0.17 Å⁻¹. Sodium caprate Form B comprises a WAXS peak at a region of about 0.23 Å⁻¹. Sodium caprate Forms C and D comprise only one peak at a region of about 0.1 to about 0.3 Å⁻¹. Sodium caprate Form C and D do not comprise a WAXS peak at a region of about 0.1 to about 0.2 Å⁻¹. Sodium caprate Forms C and D sodium caprate do not comprise WAXS peaks at about 0.12 Å⁻¹ and 0.17 Å⁻¹. Sodium caprate Form C and D comprise a WAXS peak at about 0.23 Å⁻¹

### Example 3. Small-Angle X-Ray Scattering of Sodium Caprate Forms

Small-angle X-ray scattering (SAXS) was used to further characterize the different forms of sodium caprate. Results are shown in FIGS. 4A-4D. Form A sodium caprate comprises a SAXS peak at a region of about 0.1 to about 0.15 Å⁻¹. Form A sodium caprate comprises more than one SAXS peaks at a region of about 0.12 to about 0.23 Å⁻¹. Form A sodium caprate comprises a SAXS peak at about 0.12 Å⁻¹. Form A sodium caprate comprises a SAXS peak at about 0.23 Å⁻¹. Sodium caprate Form B comprises a SAXS peak at a region of about 0.1 to about 0.2 Å⁻¹. Sodium caprate Form B comprises a SAXS peak at about 0.17 Å⁻¹. Sodium caprate Form B comprises a SAXS peak at a region of about 0.23 Å⁻¹. Forms C and D sodium caprate comprise only one peak at a region of about 0.1 Å⁻¹ to about 0.3 Å⁻¹. Forms C and D sodium caprate do not comprise a SAXS peak at 0.12 Å⁻¹.

### Example 4. Karl Fischer Titration Analysis of Sodium Caprate Forms

The Karl Fischer titration method was used to determine the water content in the different forms of sodium caprate prepared as described in Example 1. Results are summarized in Table 3. The different solid-state forms differ in the amount of water they contain. The results show that Form D sodium caprate has the highest water content, followed by Form C. Form A has water content of less than 2%.

TABLE 3
Water Content in Sodium Caprate Forms

| Form | Amount of Water (%) |
|---|---|
| Form A | <2 |
| Form B | <1 |
| Form C | 2-3 |
| Form D | 2.5-4 |

### Example 5. Compaction Properties of Sodium Caprate Forms

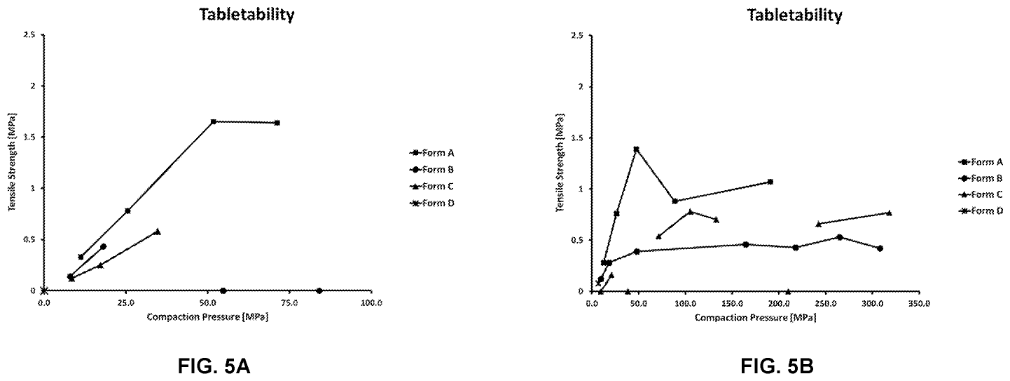

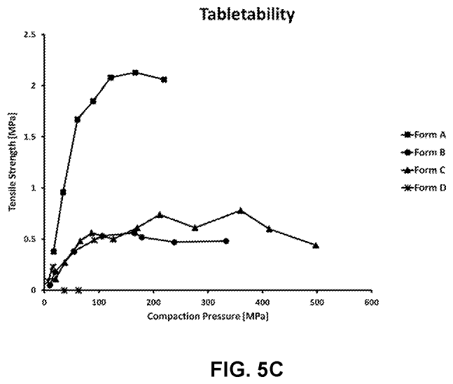

The tabletability of different particle sizes and forms of sodium caprate was obtained by measuring the tensile strength of tablets compressed using a compaction simulator. FIGS. 5A-5C show the results of tabletability with laboratory-scale amounts of sodium caprate with particle sizes of about 37 to 53 μm, of about 150 to 300 μm, and of about 600 to 710 μm, respectively.

Tablets were produced with each of the sodium caprate forms, and compaction properties were assessed. Form A exhibited the best compaction properties, e.g., pressure-dependent stickiness and no observable sticking at high pressure (above approximately 50 MPa). Form B, Form C and Form D had poor tabletability and high sticking tendency. Form C and Form D had the worst tabletability as it was not possible to produce intact compact tablets at most of the applied compaction pressures.

### Example 6. Preparation of Sodium Caprate Forms

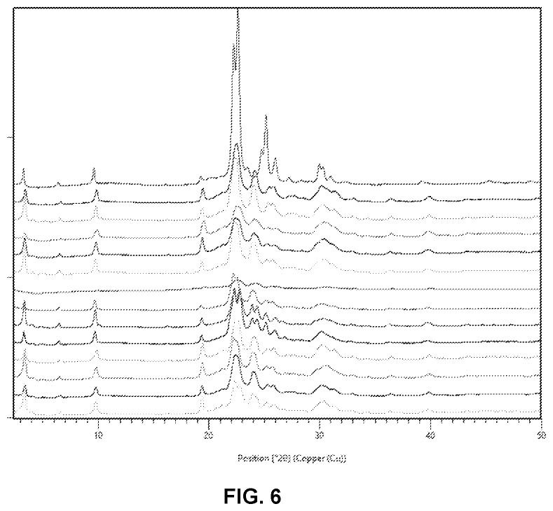

Sodium caprate was treated with different solvents and subjected to XRPD. Results in FIG. 6 show that Form C sodium caprate was formed when sodium caprate was treated with water, while predominantly Form A sodium caprate was formed when sodium caprate was treated with a non-aqueous solvent, e.g., methanol (MeOH), ethanol (EtOH), toluene, cyclohexane, dioxane, dimethylformamide (DMF), acetonitrile (ACN), tetrahydrofuran (THF), methyl isobutyl ketone (MIBK), ethyl acetate (EtOAc), and diethylether. Sodium caprate treated with dimethylsulfoxide (DMSO) resulted in an amorphous form with small amount of crystalline material.

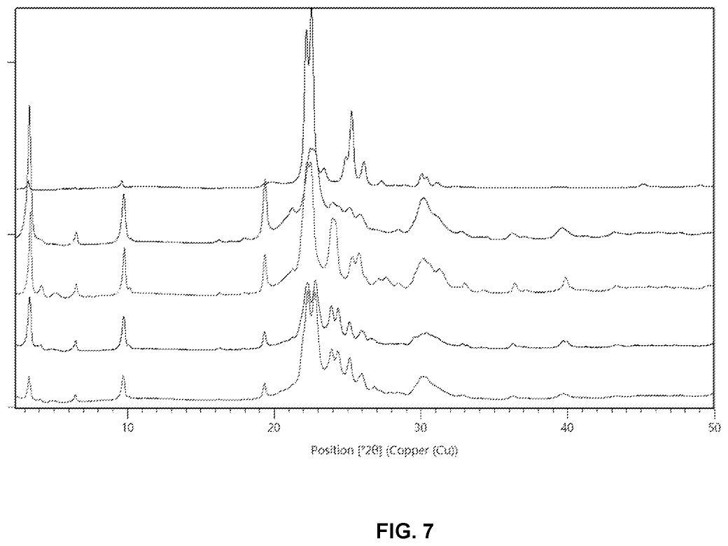

A further experiment was conducted with a smaller panel of solvents. As shown in FIG. 7, treatment with solvent containing water resulted in Form C sodium caprate, while treatment with solvent (e.g., ethanol, acetonitrile, acetone, or toluene) only resulted in predominantly Form A sodium caprate.

### Example 7. Wet Granulation of Sodium Caprate with an Alcohol or Water

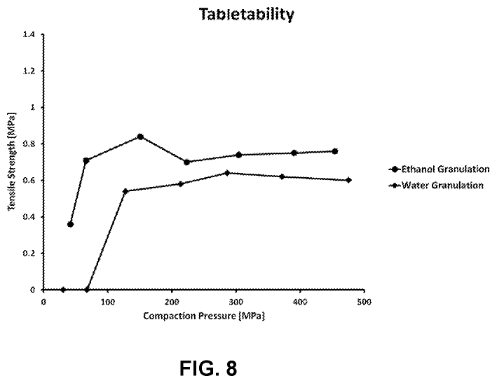

Sodium caprate was wet granulated with water and with ethanol, dried, grinded and sieved. As shown in FIG. 8, the sieved granules comprising Form A sodium caprate from ethanol granulation had superior compaction properties compared to the compaction properties of the sieved granules prepared using water granulation. Water granulated material showed extensive sticking to tableting machine punches and die. Independently on the compaction pressure applied, no sticking was observed for the granules granulated in ethanol.

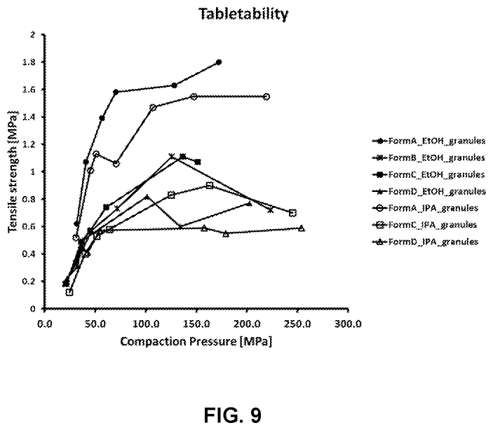

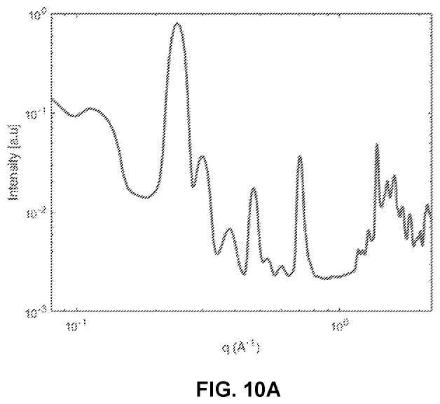

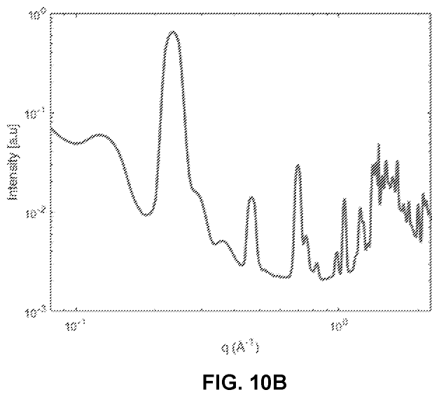

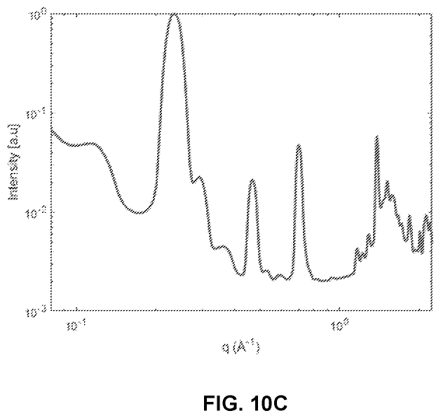

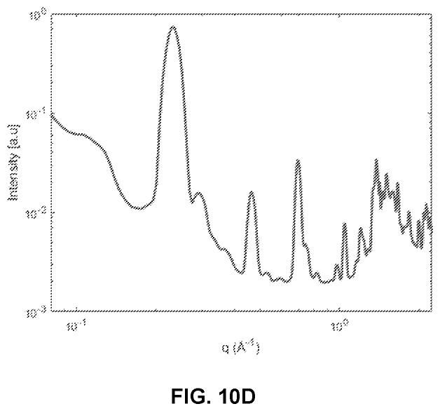

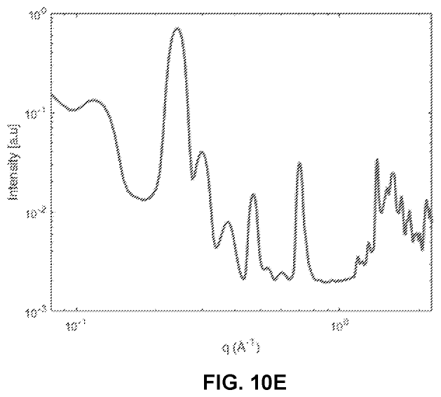

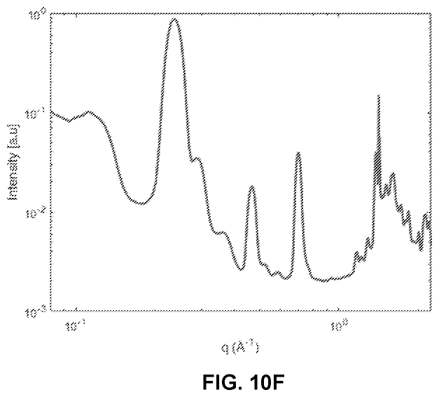

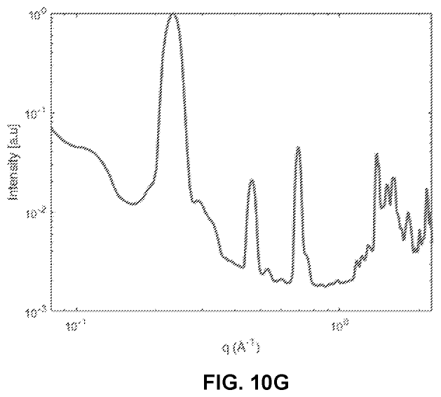

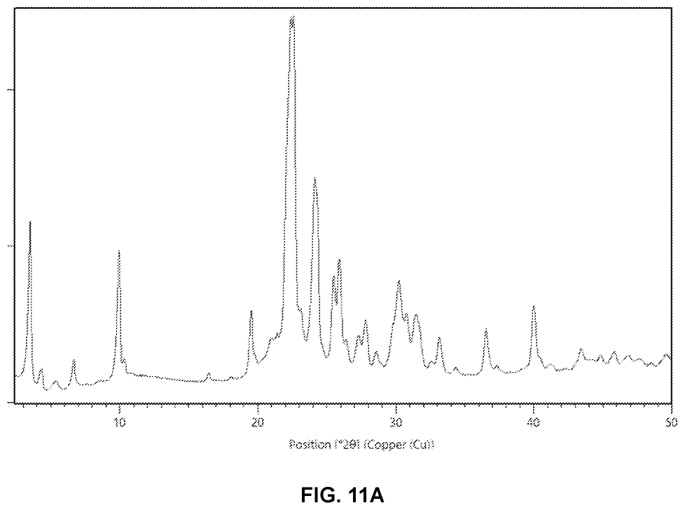

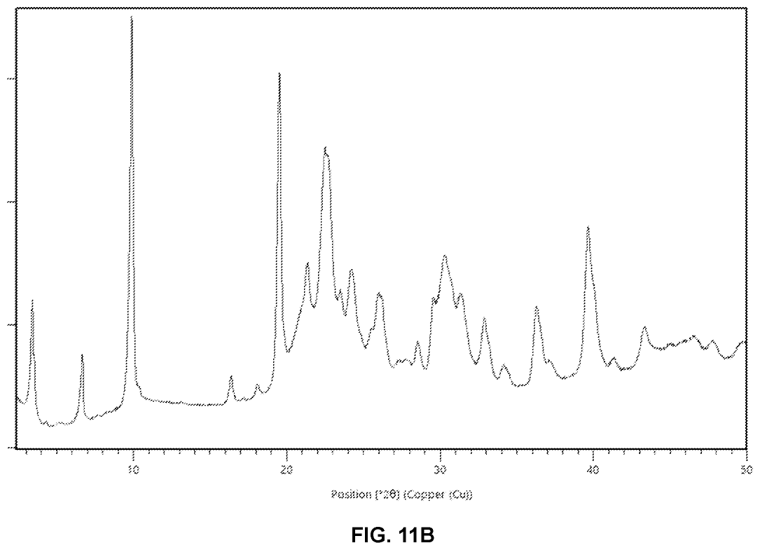

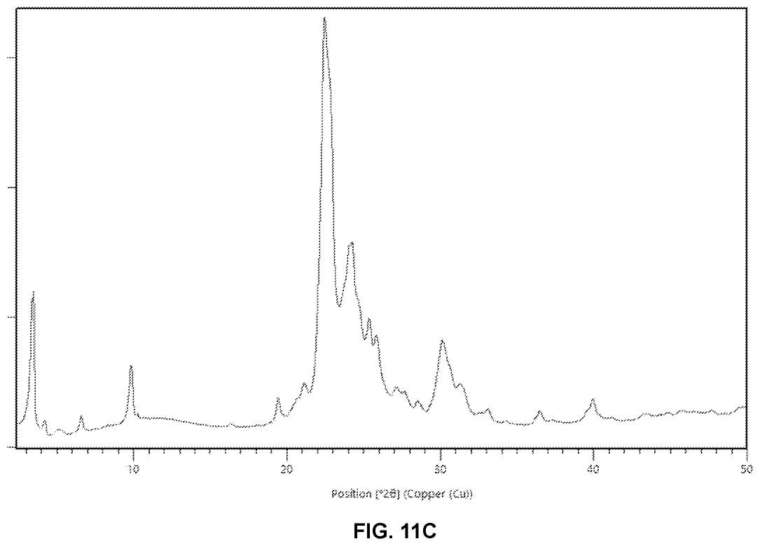

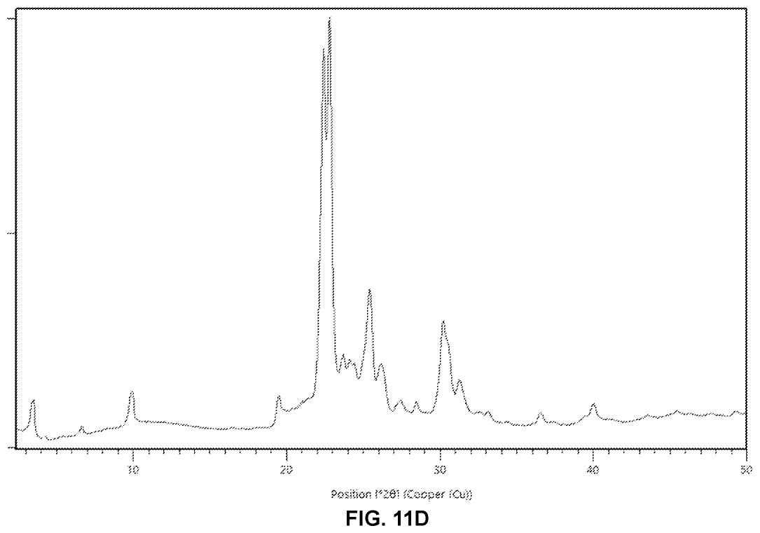

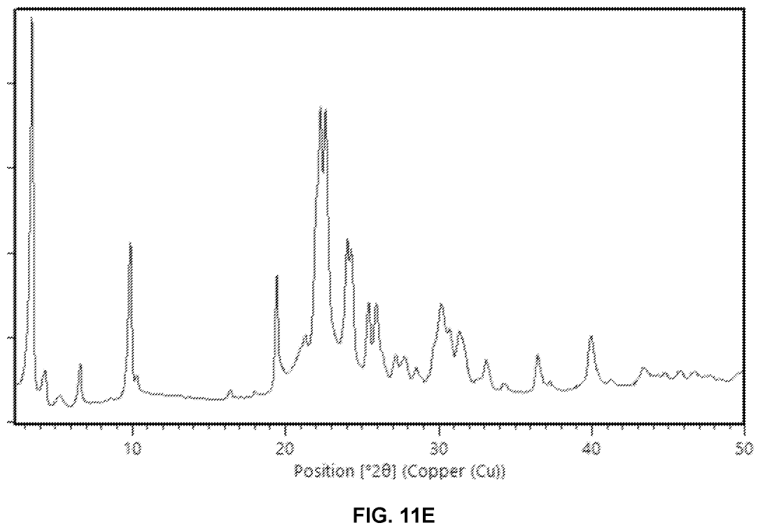

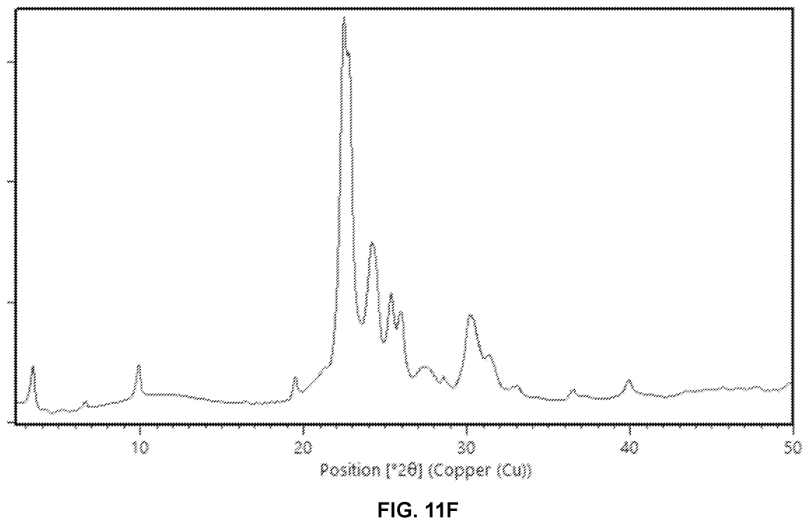

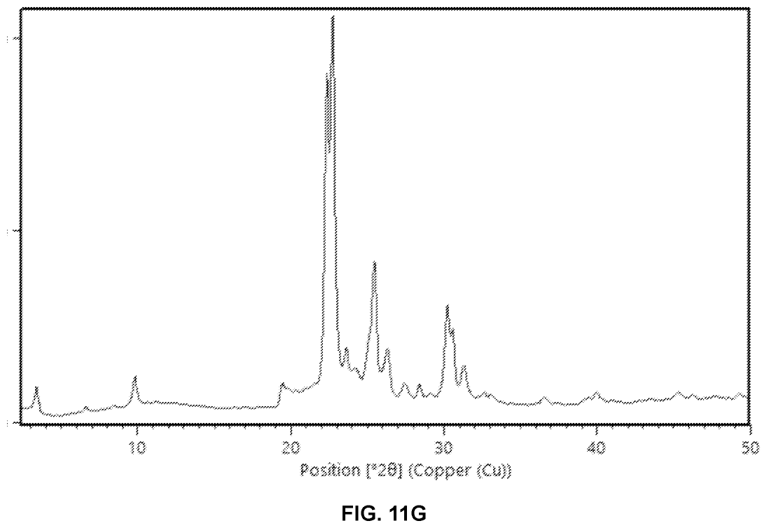

Forms A, B, C and D sodium caprate were each wet granulated with ethanol. Forms A, C and D sodium caprate were each wet granulated with 2-propanol. The wet granulated sodium caprate materials were subsequently dried and milled. After granulation with ethanol or 2-propanol it was possible to form tablets compacts of all granulated sodium caprate materials without any sticking or picking observed and with improved compaction properties, as shown in FIG. 9. WAXS analysis of the wet granulated, dried and milled sodium caprate materials showed the presence of sodium caprate Form A, as shown in FIGS. 10A-10G. XRPD analysis of the wet granulated dried and milled sodium caprate materials showed the presence of sodium caprate Form A, as shown in FIGS. 11A-11G.

### Example 8. Preparation of Tablets Comprising Sodium Caprate Form a by Direct Compression

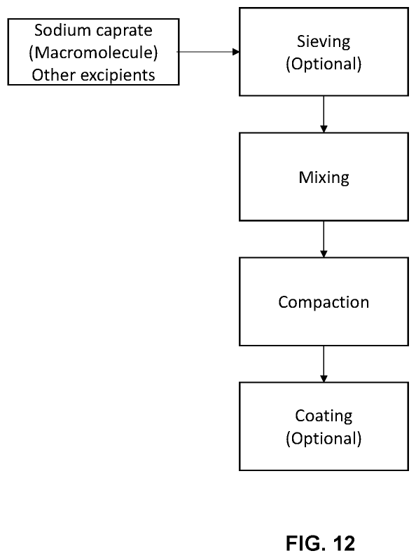

Required amount of sodium caprate Form A and mannitol was weighed into a bin blender. The components were blended. Optionally sodium stearyl fumarate were sieved through a screen, added to the dry mix above and blended. The final blend was compacted to tablets using a ten-station rotary tablet press. The process flowchart is schematically illustrated in FIG. 12.

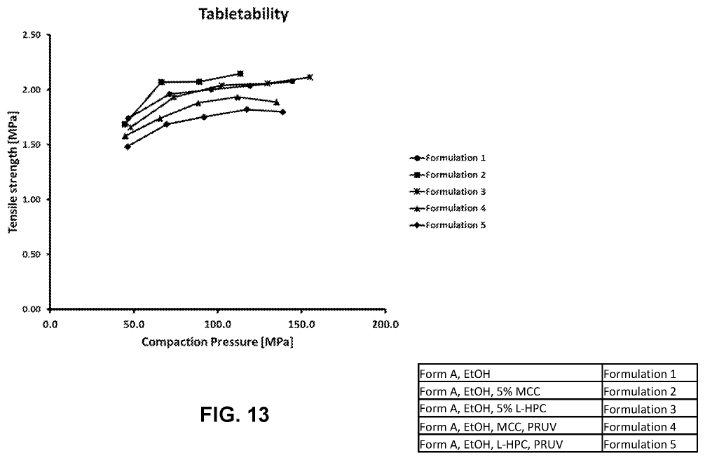

FIG. 13 shows the tabletability of sodium caprate Form A wet granulated with ethanol dried and milled and sodium caprate Form A mixed with either microcrystalline cellulose (MCC) or hydroxy propyl cellulose (L-HPC) and with and without sodium stearyl fumarate (PRUV)

### Example 9. Preparation of Tablets Comprising a Macromolecule and Form A Sodium Caprate Wet Granulated with Mannitol

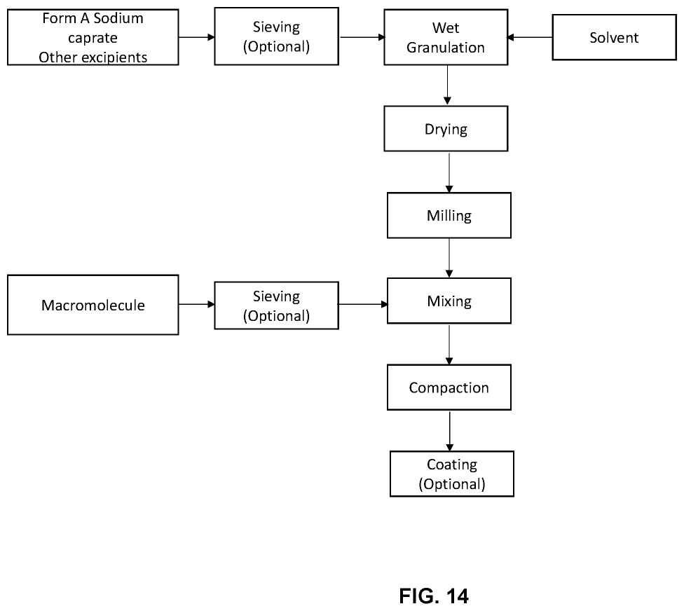

The required amount of sodium caprate Form A and mannitol will be weighed into a 60 L high shear mixer. The two components will be dry mixed for 1 minute in the high shear mixer at an impeller speed of 140 rpm. During continuous mixing in the high shear mixer, ethanol will be added until appropriate degree of granulation is reached. After granulation, the wet granules will be transferred into a fluid bed dryer and dried at an inlet temperature of 70° C. until pre-defined loss on drying of the granules is reached (<1.8%). The dried granules will then be milled through a cone mill with a screen size of 1.27 mm. The milled granules will then be blended in an 80 L diffusion mixer, with a macromolecule (through a screen size of 0.25 mm), silica colloidal hydrated (through a screen size of 0.5 mm) and microcrystalline cellulose, for 11 minutes at a rotational speed of 30 rpm. Sodium stearyl fumarate will be added (through a screen size of 0.5 mm) to the blend and mixed for an additional 3 minutes at 30 rpm. The final blended granules will be compacted to uncoated core tablets using an eight-station rotary tablet press. The uncoated tablets will then be coated with a gastric-resistant coating (sieved through a screen size 0.25 mm) in a pan coater (drum size 5 kg) at an inlet temperature of 50° C. to 70° C. The process flowchart is schematically illustrated in FIG. 14.

### Example 10: Wet Granulation of Sodium Caprate with Mannitol Using an Alcohol

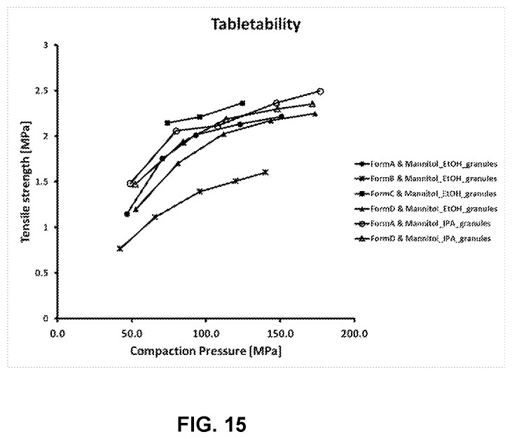

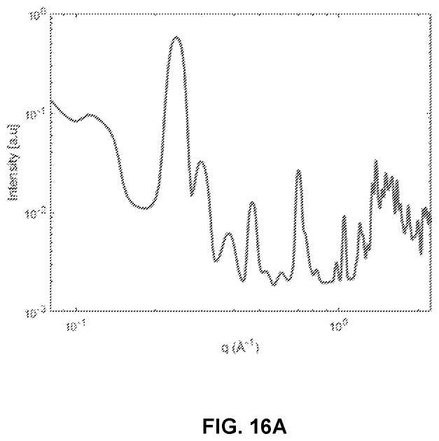

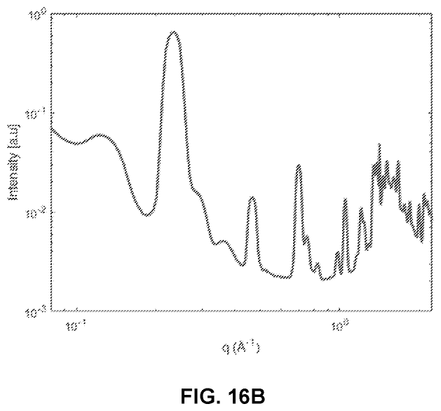

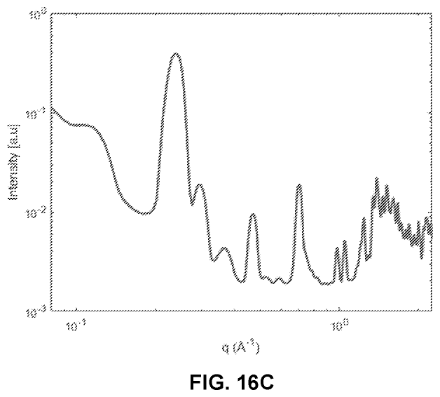

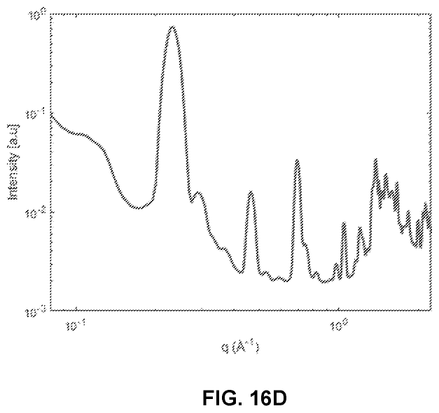

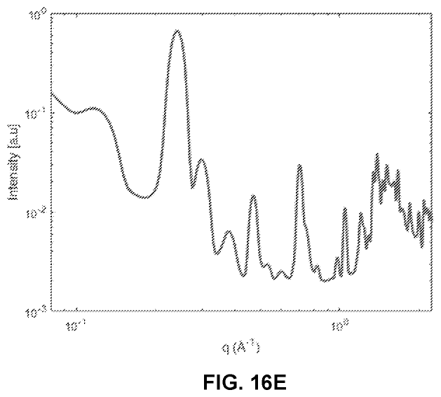

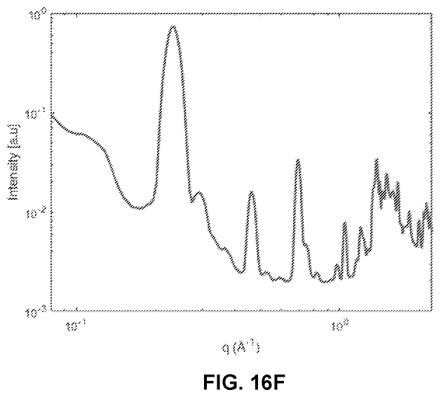

PLACEBO formulations of Forms A, B, C and D sodium caprate together with mannitol, were each subjected to wet granulation with ethanol. Forms A, C and D sodium caprate were each subjected to wet granulation with 2-propanol. The granulated materials were subsequently dried and milled. A process flowchart for the full process is schematically illustrated in FIG. 14. As shown in FIG. 15, sodium caprate combined with mannitol after granulation with ethanol or 2-propanol had improved compaction properties. WAXS analysis of the granulated (dried and milled) material showed the presence of sodium caprate Form A, as shown in FIGS. 16A-16F. XRPD analysis of the granulated (dried and milled) material showed the presence of sodium caprate Form A, as shown in FIGS. 17A-17F.

### Example 11. Preparation of Form a, Form B, Form C, and Form D Sodium Caprate

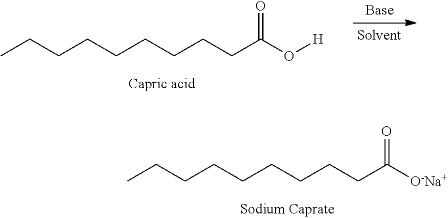

Preparation of Sodium Caprate Form A, method 1.

Methanol (234 L) and water (12.3 L) was added to pre-melted capric acid (24.6 kg, 143 mols) and sodium bicarbonate (11.0 kg, 131 mols). The reaction mixture was heated at 65±5° C. and stir for at least 16 hours. When the reaction was finished (capric acid ≤3 mg/mL) the reaction solution was filtered through a polish filter. The temperature was adjusted 50±5° C., the mixture was cooled (5° C./h) to 27±3° C. and tert-butyl methyl ether (TBME, 492 liters) was added. The temperature was adjusted to 25±5° C. and stirred for at least 12 hours. The solids were collected by centrifugation and washed with TMBE (123 liters). The solids were dried under reduced pressure at 50±5° C. for at least 12 hours (Loss on Drying, LoD≤2.0%) to afford 20.99 kg of the target product, yield 81.3%, LoD 0.6%.

Preparation of Sodium Caprate Form A, method 2

Charge ethanol (400 L, 8.0 v) to reactor A and start agitation. Charge capric acid (50.0 Kg, 1.0 eq.) to reactor A. The capric acid/ethanol solution through a microporous filter to reactor B. Charge ethanol (1100 L, 22.0 v) through a microporous filter to reactor B. Heat to 65±5° C. Charge EtONa in Ethanol solution (concentration: 20.5% w/w, 94.1 Kg, 0.96 eq.) through a microporous filter to reactor B. Heat to reflux (85±3° C.). Stir for at least 3 hours. Take sample for GC (IPC), capric acid concentration ≤0.5%. Cool to 55±5° C. over 2 hours and stir for at least 6 h. Control the cooling rate to 3-5° C./h; cool the mixture temperature to 45±5° C., continue stir for at least 6 hours. Control the cooling rate 3-5° C./h; cool the mixture temperature to 30±5° C., continue stir for at least 10 hours. Isolation; centrifuge under a nitrogen atmosphere, wash the cake once with ethanol (3.0 v). Dry the cake under vacuum at 50±5° C. for at least 12 hours. (mix the material every 2 to 4 h.). Sample every 4 to 6 hours, until LoD≤2.0%. Collect the crude product. 44.50 kg, LoD: 1.1%. Sieve the material with a 20 mesh (800 μm) sieve. 43.32 kg of the product was obtained.

Preparation of Sodium Caprate Form A, method 3

Capric acid (15 g) was dissolved in 150 ml EtOH/water (12%-vol) at 65° C. with stirring (500 rpm) until dissolution occurred. Sodium ethoxide in EtOH solution (77 ml, 1.1 M, 1.0 eq) was added and stirred for 30 min (final water content 8%). A seed suspension was made by suspending sodium caprate Form A (500 mg, 3%-wt) in TBME (225 ml) at 50° C. with stirring (450 rpm). The capric acid solution was added to the seed suspension dropwise over 2.5 h at the midpoint between the impeller and the wall. After the addition further sodium ethoxide solution in EtOH (1.1 M, 0.3 eq) was added 1 h and stirred for 30 min. The sample was then cooled to 25° C. at 0.1° C./min then temperature cycled (25-40° C.; two cycles cooling rate 0.1° C./min, heating rate 2° C./min) with an end temperature of 25° C. Isolation was done by filtration under suction under a cone of nitrogen, then washed with chilled TBME (75 ml). The sample was dried in a vacuum oven at 40° C. overnight.

### Preparation of Sodium Caprate Form B

Sodium caprate powder was transferred in a reactor and powder was heated under flow of nitrogen gas to 280° C. The melt obtained upon heating was continuously stirred. Heating was stopped when all material was liquid. The liquid in the reactor was allowed to cool down to room temperature overnight under flow of nitrogen gas. The resultant solid sodium caprate Form B was collected from the sealed reactor

### Preparation of Sodium Caprate Form C

Capric acid (1516.5 g, 8.80 mols, 1.0 eq) was dissolved in ethanol (26 liters) and heated to 75-80° C. At temperature, sodium hydroxide solution (3683 g, 9.46% w/w, 8.71 mols, 0.99 eq) was charged, over approximately 30 minutes to the vessel keeping the temperature within the range of 70-80° C. After addition was complete, the mixture was stirred for 15 minutes before being cooled to 60° C. and polished filtered into a second vessel at 60° C. and then cooled to 52° C. TBME (15 liters) was then charged to the reaction mixture keeping the temperature within the range of 45-52° C. After the addition, the batch was cooled to 37.5° C. and then seeded (1.5 g, sodium caprate). The batch was cooled to 36° C. and then stirred for 1 hour between 34-36° C. During this time the reaction mixture became translucent, indicating further precipitation of the product. The batch was cooled to 7-14° C. for 15-17 hours, collected by filtration and dried under reduced pressure at 50° C. to constant weight (1330 g, 77.8% yield, LoD 1.69%).

### Preparation of Sodium Caprate Form D

Charge THF (7 L, 4.5 v) to reactor A and start stirring. Charge sodium caprate (Form A, 1.56 Kg, 1.0 eq.) to reactor A. Charge water (936 ml, 0.6 v/w.) to reactor A at 20-30° C. Heat to 65° C., stir for 2 h. (A clear solution observed.) Cool to 20° C. in 5 h. Stir for 38 h at 20° C. Take sample for XRPD (Form D) Filter and wash the cake once with THF (780 ml, 0.5 v). Dry the cake under vacuum at 45±5° C. for at least 16 h. Collect the crude product, 952 g, yield: 61%.

### Example 12. Preparation of Tablets Comprising MEDI7219 Peptide or MALAT1 ASO or PLACEBO and Form A Sodium Caprate Wet Granulated, Formulation 1

1200 g of sodium caprate Form A was added to an intensive blending equipment and dry mixed, impeller speed 250 rpm and chopper speed 1500 rpm, for 1 min before addition of 600 g of absolute ethanol (30 mL/min). After complete addition the resulting material was mixed for an additional 1 min. The material was dried overnight in a fume cupboard at ambient temperature until <LoD 1.5%. The dried material was milled using a Quadro comil equipped with a 0.061′ grater screen and an angular impeller at 2000 rpm.

MEDI7219 Peptide-Sodium caprate Form A (93 g) wet granulated as described in Example 12, micro crystalline cellulose (Avicel PH-102, 5.0 g) and MEDI7219 Peptide (10 mg) were passed through a 500 μm sieve and pre-blended by hand with a spoon in a metal vessel for 1 min. Additional blending was done in a turbula mixer for 8 min. Sodium stearyl fumarate (PRUV®, 2 g) was passed through a 250 μm sieve and pre-blended by hand with a spoon for 1 min. Addition blending was done in a turbula mixer for 2 minutes. The material was compacted in a tablet press.

MALAT1 ASO—Sodium caprate Form A (93 g) wet granulated as described in Example 12, micro crystalline cellulose (Avicel PH-102, 5 g), and MALAT 1 ASO (20 mg) were passed through a 500 μm sieve and pre-blended by hand with a spoon in a 1 L metal vessel for 1 min. Additional blending was done in a turbula mixer for 8 min. Sodium stearyl fumarate (PRUV®, 2 g) was passed through a 250 μm sieve and pre-blended by hand with a spoon for 1 min. Addition blending was done in a turbula mixer for 2 minutes. The material was compacted in a tablet press.

PLACEBO—Sodium caprate Form A (93 g) wet granulated as described in Example 12 and micro crystalline cellulose (Avicel PH-102, 5 g) were passed through a 500 μm and pre-blended by hand with a spoon in a 1 L metal vessel for 1 min. Additional blending was done in a turbula mixer for 8 min. Sodium stearyl fumarate (PRUV®, 2 g) was passed through a 250 μm sieve and pre-blended by hand with a spoon for 1 min. Addition blending was done in a turbula mixer for 2 minutes. The material was compacted using a single punch equipment, equipped with an oblong punches, 16×8 mm to in a tablet press to a tablet tensile strength of 0.8 MPa.

PLACEBO—Sodium caprate Form A (87 g) wet granulated as described in Example 12, micro crystalline cellulose (Avicel PH-102, 10 g) and silicon dioxide (Syloid 244FP, 1 g) were passed through a 500 μm and pre-blended by hand with a spoon in a 1 L metal vessel for 1 min. Additional blending was done in a turbula mixer for 8 min. Sodium stearyl fumarate (PRUV®, 2 g) was passed through a 250 μm sieve and pre-blended by hand with a spoon for 1 min. Addition blending was done in a turbula mixer for 2 minutes. The material was compacted using a single punch equipment, equipped with an oblong punches, 16×8 mm to in a tablet press to a tablet tensile strength of 1.4 MPa.

PLACEBO—Sodium caprate Form A (92 g) wet granulated as described in Example 12, micro crystalline cellulose (Avicel PH-102, 5 g) and silicon dioxide (Syloid 244FP, 1 g) were passed through a 500 μm and pre-blended by hand with a spoon in a 1 L metal vessel for 1 min. Additional blending was done in a turbula mixer for 8 min. Sodium stearyl fumarate (PRUV®, 2 g) was passed through a 250 μm sieve and pre-blended by hand with a spoon for 1 min. Addition blending was done in a turbula mixer for 2 minutes. The material was compacted using a single punch equipment, equipped with an oblong punches, 16×8 mm to in a tablet press to a tablet tensile strength of 1.2 MPa.

### Example 13. Preparation of PLACEBO Tablets Comprising Form A Sodium Caprate Wet Granulated with Mannitol, Formulation 2

Sodium caprate Form A (7700 g) and mannitol (Pearlitol 100SD, 3300 g) were weighed into a 60 L high shear mixer. The two components were dry mixed for 3 minutes with an impeller speed of 140 rpm. During continuous mixing ethanol (4050 g) was added until appropriate degree of granulation was reached. After granulation, the wet granules were transferred into a fluid bed dryer and dried at an inlet temperature of 70° C. until pre-defined loss on drying of the granules was reached (<1.8%). The dried granules were milled through a cone mill with a screen size of 1.27 mm. LoD after milling was 0.5%-w/w.

The dried granules from above (47.8 g), micro crystalline cellulose (Avicel PH-102, 2.5 g), silicon dioxide (Syloid 244FP, 0.5 g) were passed through a 250 μm sieve, were pre-blended in a 1 L metal vessel for 1 min by hand with a spoon, additional blending was done in a turbula mixer for 10 min. Sodium stearyl fumarate (1.0 g) was added to about 10 g the resulting mixture above and pre-blended for 1 min by hand with a spoon, the remaining amount of the mixture above was added and additional blending was done in a by hand with a spoon for 2 min. The material was compacted “as is” in a tablet press with a tensile strength of 2.0 MPa.

### Example 14. Dissolution Profiles of MALAT1 ASO and MEDI7219 Peptide

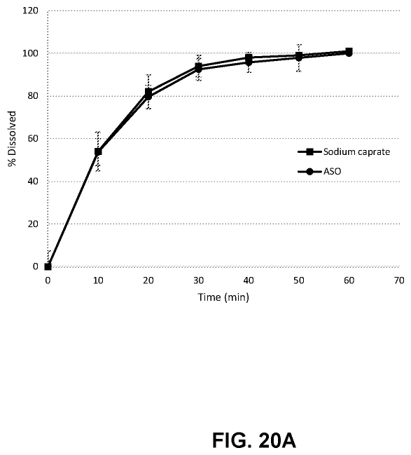

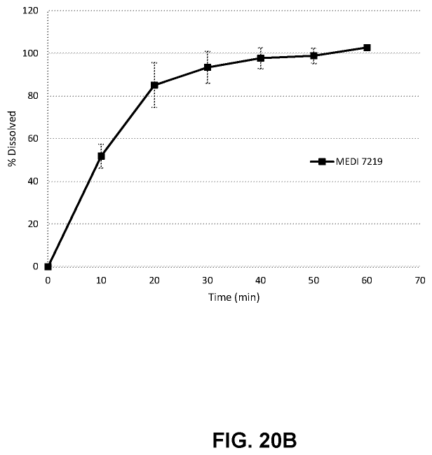

The dissolution profiles of tablets comprising Form A sodium caprate and an ASO, prepared according to Example 12, Formulation 1, are illustrated in FIG. 20A. The graph shows that there is a rapid and concomitant release (dissolution) of the ASO (MALAT1 in this example) and Form A sodium caprate from tablets at pH 6.8 phosphate buffer at 37° C. (mimicking the small intestinal condition). This allows for maximum absorption enhancement. The dissolution profile of tablets comprising Form A sodium caprate and the MEDI7219 Peptide is illustrated in FIG. 20B. The graph shows that there is a rapid release (dissolution) of the polypeptide (MEDI7219 in this example) from tablets at a pH 6.8 phosphate buffer at 37° C. (mimicking the small intestinal condition).

### Example 15. Preparation of Spherical Agglomerates from Form A Sodium Caprate

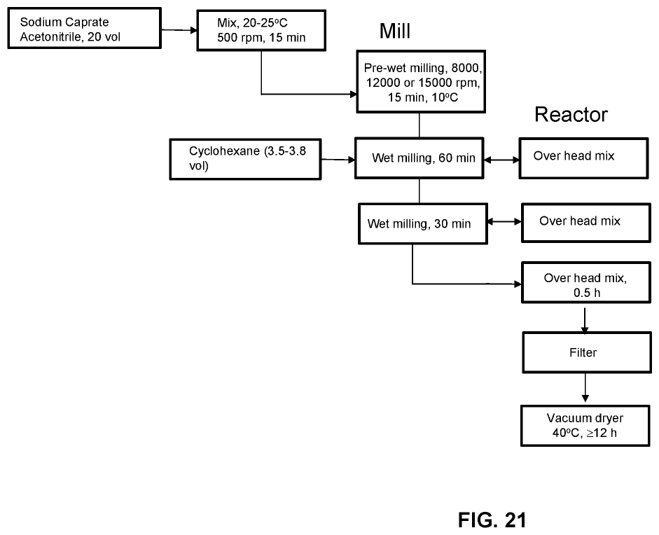

Sodium caprate Form A particles prepared from Example 11, methods 3, were pre-wetted with acetonitrile (dispersant liquid to solid ratio: 1:20) with stirring at 500 rpm for about 15 min to ensure a homogeneous slurry. The slurry was wet milled for 15 min at 10° C. (8000, 12000, 15000 rpm) using an IKA Magic Lab mill to reduce the particle size. The bridging liquid, cyclohexane, 1:3.5 to 1:3.8 liquid to solid ratio, was added over 60 min. After complete addition the high shear mixing is continued for an additional 30 minutes followed by 30 min of overhead stirring. The spherical agglomerates are collected by filtration under dried in a vacuum oven over night at 40° C. By variation of the amount of bridging liquid and high shear wet milling rotor speed agglomerates with a mean sizes (D(50)) between 230 μm and 1330 μm are produced. A process flowchart for the full process is schematically illustrated in FIG. 21.

What is claimed is:

1. A method of making an oral tablet, the method comprising
a) mixing a macromolecule with sodium caprate to form a mixture;
b) direct compressing the mixture to form a tablet;
wherein the sodium caprate is Form A sodium caprate characterized by a wide-angle X-ray scattering (WAXS) spectrum which includes a peak at a region of 0.1 to 0.15 Å⁻¹.

2. The method of claim 1, wherein the sodium caprate is Form A sodium caprate characterized by a wide-angle X-ray scattering (WAXS) spectrum which includes a peak at 0.12 and 0.23 Å⁻¹.

3. The method of any one of claim 1 or 2, wherein the sodium caprate is Form A sodium caprate characterized by a small-angle X-ray scattering (SAXS) spectrum which includes a peak at 0.12 and 0.23 Å⁻¹.

4. The method of any one of claims 1 to 3, wherein the sodium caprate is Form A sodium caprate characterized by a X-ray powder diffraction (XRPD) spectrum which includes a peak at 4° 2θ.

5. The method of any one of claims 1 to 4, wherein the sodium caprate has a water content of less than about 3.5% as measured by Karl Fischer titration.

6. The method of any one of claims 1 to 5, wherein the sodium caprate has a water content of less than about 2.0% as determined by Karl Fischer titration

7. The method of any one of claims 1 to 6, wherein the sodium caprate has a water content of less than about 1.5% as determined by Karl Fischer titration.

8. The method of any one of claims 1 to 7, wherein the sodium caprate of step (a) is sieved or milled to obtain a desired particle size before the direct compression of (b) to form a tablet.

9. The method of any one of claims 1 to 8, wherein the mixture of step (a) is sieved or milled to obtain a desired particle size before the direct compression of (b) to form a tablet.

10. The method of any one of claims 1 to 9, wherein the sodium caprate has a particle size ranging from about 35 μm to about 1300 μm before step (b).

11. The method of claim 10, wherein the sodium caprate has a particle size ranging from about 100 μm to about 850 μm.

12. The method of claim 11, wherein the sodium caprate has a particle size ranging from about 250 μm to about 750 μm.

13. The method of any one of claims 1 to 12, wherein after the tablet compression of (b), a coating is formed on the tablet.

14. A method of making an oral tablet, the method comprising
a) Wet granulating sodium caprate in a non-aqueous organic solvent to form granules;
b) Drying and milling the granules
c) Mixing the resulting granules with the macromolecules and other excipients
d) Compressing the mixture to form a tablet;
wherein the sodium caprate after step b) is Form A sodium caprate characterized by a wide-angle X-ray scattering (WAXS) spectrum which includes a peak at a region of 0.1 to 0.15 Å⁻¹ and/or by an X-ray powder diffraction (XRPD) spectrum which includes a peak at 4° 2θ.

15. A method of making an oral tablet, the method comprising
a) Wet granulating sodium caprate together with other excipients in a non-aqueous organic solvent to form granules;
b) Drying and milling the granules
c) Mixing the resulting granules with the macromolecules and optionally additional excipients; and
d) Compressing the mixture to form a tablet;
wherein the sodium caprate after step b) is Form A sodium caprate characterized by an X-ray powder diffraction (XRPD) spectrum which includes a peak at 4° 2θ.

16. A method of making an oral tablet, the method comprising
a) Wet granulating sodium caprate together with a macromolecule and possibly other excipients in a non-aqueous organic solvent to form granules;
b) Drying and milling the granules;
c) Mixing the resulting granules with the other excipients; and
d) Compressing the mixture to form a tablet;
wherein the sodium caprate after step b) is Form A sodium caprate characterized by an X-ray powder diffraction (XRPD) spectrum which includes a peak at 4° 2θ.

17. The method of any one of claims 1 to 16, wherein the compressing is performed at greater than 10 MPa.

18. The method of any one of claims 1 to 17, wherein the compressing is performed at 15 MPa to 500 MPa.

19. The method of any one of claims 1 to 18, wherein the compressing is performed at 25 MPa to 300 MPa.

20. The method of any one of claims 1 to 19, wherein the compressing is performed at 50 MPa to 200 MPa.

21. The method of any one of claims 1 to 20, wherein the mixing of (a) further comprises a polyol.

22. The method of any one of claims 1 to 21, wherein the polyol is chosen from sorbitol, mannitol, maltitol, xylitol and combinations thereof.

23. The method of any one of claims 1 to 22, wherein the polyol is mannitol.

24. The method of any one of claims 1 to 23, wherein the macromolecule is about 0.1% to about 12% by weight of the tablet.

25. The method of any one of claims 1 to 24, wherein the sodium caprate is about 30% to about 99% by weight of the tablet.

26. The method of any one of claims 1 to 25, wherein the polyol is about 0.001% to about 50% by weight of the tablet.

27. The method of any one of claims 1 to 26, wherein the macromolecule is a peptide or an oligonucleotide.

28. The method of any one of claims 1 to 27, wherein the macromolecule is an oligonucleotide.

29. The method of any one of claims 1 to 28, wherein the macromolecule is an oligonucleotide of SEQ ID NO: 1.

30. The method of any one of claims 1 to 29, wherein the oral tablet has a tablet hardness tensile strength of about 0.5 MPa to about 5 MPa.

31. The method of any one of claims 1 to 30, wherein the oral tablet has a tablet hardness tensile strength of about 0.75 MPa to about 3 MPa.

32. The method of any one of claims 1 to 31, wherein the oral tablet has a tablet hardness tensile strength of about 1.0 MPa to about 2.5 MPa.

33. A method of making an oral tablet, the method comprising
a) sieving sodium caprate to obtain sodium caprate particles having a particle size of about 35 μm to about 1100 μm
b) mixing the sodium caprate particles with a macromolecule to form a mixture;
c) direct compressing the mixture of to form a tablet;
wherein the sodium caprate is Form A sodium caprate characterized by a wide-angle X-ray scattering (WAXS) spectrum which includes a peak at a region of 0.1 to 0.15 Å⁻¹ and/or by an X-ray powder diffraction (XRPD) spectrum which includes a peak at 4° 2θ and wherein the method does not comprise a wet granulation step, a drying step or a milling step after the mixing of step (a).

34. A tablet made by the method of any one of claims 1 to 33.

35. A composition comprising a macromolecule and sodium caprate, wherein the sodium caprate is Form A sodium caprate characterized by a wide-angle X-ray scattering (WAXS) spectrum which includes a peak at a region of 0.1 to 0.15 Å⁻¹ and/or by an X-ray powder diffraction (XRPD) spectrum which includes a peak at 4° 2θ.

36. The composition of claim 35, further comprising a polyol.

37. The composition of claim 36, wherein the polyol is mannitol.

38. The composition of any one of claims 35 to 37, wherein the macromolecule is about 0.1% to about 12% by weight of the tablet.

39. The composition of any one of claims 35 to 38, wherein the sodium caprate is about 30% to about 99% by weight of the tablet.

40. The composition of any one of claims 35 to 39, wherein the polyol is about 0.001% to about 50% by weight of the tablet.

41. The composition of any one of claims 35 to 40, wherein the macromolecule is a peptide or an oligonucleotide.

42. The composition of any one of claims 35 to 41, wherein the macromolecule is an oligonucleotide.

43. The composition of any one of claims 35 to 42, wherein the macromolecule is an oligonucleotide of SEQ ID NO: 1.

44. The composition of any one of claims 35 to 45, wherein the composition is in the form of an oral tablet, and the oral tablet has a tablet hardness tensile strength of above 0.5 MPa.

45. The composition of claim 44, wherein the oral tablet has a tablet hardness tensile strength of about 0.5 MPa to about 5 MPa.
